(19) United States
(12) Patent Application Publication
(10) Pub. No.: US 2024/0336961 A1
(43) Pub. Date: Oct. 10, 2024

(54) USE OF DESTABILIZING NUCLEOTIDE TRIPHOSPHATES TO REDUCE AMPLICON FOLDING AND Tm

(71) Applicant: Cepheid, Sunnyvale, CA (US)

(72) Inventors: Sergey G. Lokhov, Seattle, WA (US); Michael DeClue, Sunnyvale, CA (US); Cameron Nakatani, Sunnyvale, CA (US)

(73) Assignee: Cepheid, Sunnyvale, CA (US)

(21) Appl. No.: 18/583,105

(22) Filed: Feb. 21, 2024

Related U.S. Application Data
(60) Provisional application No. 63/447,815, filed on Feb. 23, 2023.

(51) Int. Cl. C12Q 1/6848; C12Q 1/6855

(52) U.S. Cl. CPC C12Q 1/6848; C12Q 1/6855

(57) ABSTRACT

Described herein are methods and compositions that provide highly efficient primer annealing and denaturation during nucleic acid amplification. The basic method employs destabilizing nucleotide triphosphates (dNTPs) in amplification reaction mixtures. The incorporation of such dNTPs into the amplicon produced upon amplification lowers the Tm of the amplicon, promoting denaturation/strand separation for the next round of primer annealing.

### CROSS-REFERENCE TO RELATED APPLICATIONS

This application claims the benefit of U.S. provisional application No. 63/447,815, filed Feb. 23, 2023, which is hereby incorporated by reference in its entirety.

### STATEMENT AS TO RIGHTS TO INVENTIONS MADE UNDER FEDERALLY SPONSORED RESEARCH AND DEVELOPMENT

Not applicable.

### SEQUENCE LISTING

The instant application contains a Sequence Listing which has been submitted electronically in XML file format and is hereby incorporated by reference in its entirety. Said XML copy, created on Jun. 7, 2024, is named CPHDP023US_SL_rev.xml and is 27,263 bytes in size.

### FIELD

The methods and compositions described herein relate generally to the area of nucleic acid amplification. In particular, described herein are methods and compositions for reducing amplicon folding and Tm.

### BACKGROUND

A wide variety of nucleic acid amplification methods are available, and many have been employed in the implementation of sensitive diagnostic assays based on nucleic acid detection. Polymerase chain reaction (PCR) remains the most widely used DNA amplification and quantitation method. However, standard PCR has several limitations. Standard PCR amplification can only achieve a less-than-two-fold increase of the amount of target sequence at each cycle. It is still relatively slow. In addition, the sensitivity of this method is typically limited, making it difficult to detect target that may be present at only a few molecules in a single reaction.

Efforts to increase amplification sensitivity and efficiency, without sacrificing specificity, have included the use of unique primer sets to achieve greater-than-base 2 amplification (U.S. Pat. No. 10,273,534) and the incorporation of modified bases that pair preferentially with their unmodified complements in primers (U.S. Patent Pub. No. 2020/0239878).

Nucleic acid amplification typically includes multiple cycles of the sequential procedures of: annealing at least two primers with a complementary or substantially complementary sequences in a target nucleic acid; synthesizing at least one strand of nucleotides in a template-dependent manner using a polymerase; and separating the strands of the newly-formed nucleic acid duplex to generate template for subsequent cycles of strand synthesis.

Amplification can comprise thermocycling or can be performed isothermally. Isothermal amplification typically requires the use of a nucleic acid polymerase that has strand displacement activity and/or some other means to effect strand separation. Thermocycling is standardly carried out by subjecting a PCR reaction mixture to three temperatures per cycle in the following sequence: denaturation, usually at about 95° C.; annealing, usually at about 5° C. below the Tm of the primers; and extension (e.g., at about 72° C.). Some methods simplify this temperature/time course to two temperatures per cycle. For example, U.S. Pat. No. 9,428,781 describes “oscillating amplification” in which a two-temperature cycle includes an upper temperature and a lower temperature that differ by no more than 20° C.

In standard PCR, each cycle produces one new nucleic acid duplexes from a single target nucleic acid duplex, yielding two nucleic acid duplexes, and standard PCR is thus understood as “base 2” PCR. With unique primer designs, base 3, producing three duplexes per cycle, and base 6, producing 6 duplexes per cycle can be achieved, reducing the time-to-result (TTR).

### SUMMARY

One of the key issues hindering all fast PCR applications is inaccessibility of folded amplicon structures and high melting temperature (Tm) amplicons that reduce primer and probe hybridization rate leading to inefficient PCR.

Described herein are kits and methods based on the use of at least one destabilizing nucleotide triphosphate in an amplification mixture to reduce the Tm of the amplicon produced upon amplification and/or reduce amplicon secondary structure. This, in turn, facilitates denaturation/strand separation and the next round of primer annealing, making amplification faster and more efficient.

Various embodiments contemplated herein may include, but need not be limited to, one or more of the following:

Embodiment 1: A kit comprising:
at least one destabilizing dNTP; and
a primer pair for amplifying a target nucleic acid in the presence of the at least one destabilizing dNTP, wherein each primer:
   comprises no more than four, three, two, or one canonical bases that hybridize to the base corresponding to the destabilizing dNTP; and
   optionally comprises a modification to increase the stability of a duplex formed upon hybridization, relative to an unmodified primer.

Embodiment 2: The kit of embodiment 1, wherein the modification is that each primer comprises the at least one stabilizing base.

Embodiment 3: The kit of embodiment 1, wherein the modification is that each primer comprises one or more peptide nucleic acid, one or more locked nucleic acid, and one or more minor groove binder.

Embodiment 4: The kit of embodiment 1 or embodiment 2, wherein the at least one destabilizing dNTP is provided in a mixture of destabilizing dNTP with the canonical dNTP corresponding to the destabilizing dNTP in a ratio of greater than about 1:1, optionally greater than about: 5:1, 10:1, 15:1, 20:1, 25:1, 30:1, or 35:1 destabilizing dNTP: canconical dNTP.

Embodiment 5: The kit of embodiment 1 or embodiment 2, wherein the kit does not comprise the canonical dNTP corresponding to the destabilizing dNTP.

Embodiment 6: The kit of any one of embodiments 1-5, wherein each primer has a Tm of from about 63 degrees C. to about 67 degrees C., optionally about 65 degrees C., when hybridized to a complementary sequence of canonical bases.

Embodiment 7: The kit of any one of embodiments 1-6, wherein the primer pair defines an amplicon that has a Tm of from about 60 degrees C. to about 75 degrees C., optionally about 70 degrees C., when measured after amplification and incorporation of the at least one destabilizing base.

Embodiment 8: The kit of any one of embodiments 1-7, wherein the primer pair defines an amplicon that has a G-C content of less than about 50%, optionally less than about 40%.

Embodiment 9: The kit of any one of embodiments 1-8, wherein the primer pair defines an amplicon that has a length of about 50 bases to about 100 bases, optionally about 65 bases to about 85 bases.

Embodiment 10: The kit of any one of embodiments 1-9, wherein the kit comprises a probe.

Embodiment 11: The kit of embodiment 10, wherein the probe comprises no more than six, five, four, three, or 2 canonical bases that hybridize to the base corresponding to the destabilizing dNTP.

Embodiment 12: The kit of embodiment 10 or embodiment 11, wherein the probe comprises at least one stabilizing base.

Embodiment 13: The kit of embodiment 2 or embodiment 12, wherein

each primer comprises at least 2, optionally 3 or 4 stabilizing bases, and/or each probe comprises at least 2, optionally 3, 4, or 5 stabilizing bases.

Embodiment 14: The kit of any one of embodiments 2, 12, and 13, where the stabilizing base is selected from 2-aminoadenine, C-5 propynyl-dC, C-5 propynyl-dU, 5-methyl-dC, and a locked nucleic acid base.

Embodiment 15: The kit of any one of embodiments 10-14, wherein the probe has a Tm of from about 60 degrees C. to about 80 degrees C., optionally about 70 degrees C., when hybridized to a complementary sequence of canonical bases.

Embodiment 16: The kit of any preceding embodiment, wherein the kit comprises one or more additional primer pairs.

Embodiment 17: The kit of embodiment 16, wherein each primer pair in the kit has the same characteristics as recited for the primer pair for amplifying a target nucleic acid in the presence of the at least one destabilizing dNTP.

Embodiment 18: A method of selecting and/or designing a target nucleic acid from which an amplicon is produced upon nucleic acid amplification in the presence of a destabilizing dNTP, a primer pair capable of producing the amplicon, and an optional probe capable of detecting the amplicon, wherein the amplicon, primers, and probe are thermally balanced to promote amplification of an amplicon having a lower Tm than that of a double-stranded form of the target nucleic acid, prior to amplification, wherein the method comprises:
selecting the target nucleic acid sequence, primers, and optional probe so that destabilizing bases are incorporated into the amplicon outside of primer and/or probe target sequences in the amplicon;
if one or more primers will face one or more destabilizing bases in the amplicon, modifying the primer to increase the stability of a duplex formed upon hybridization, relative to an unmodified primer; and/or
if a probe that will face one or more destabilizing bases is included, modifying the probe to increase the stability of a duplex formed upon hybridization.

Embodiment 19: A method for amplifying a target nucleic acid using the components of the kit of any one of embodiments 1-17, wherein the method comprises contacting the primers with sample nucleic acids under conditions suitable for amplification, such conditions comprising the presence of the at least one destabilizing dNTP, wherein the amplification produces an amplicon that has a lower Tm than that of a double-stranded form of the target nucleic acid, prior to amplification.

Embodiment 20: The method of embodiment 19, wherein the destabilizing dNTP is present in an amplification reaction mixture a ratio of greater than about 1:1, optionally about: 5:1, 10:1, 15:1, 20:1, 25:1, 30:1, or 35:1 destabilizing dNTP: canonical dNTP.

Embodiment 21: The method of embodiment 19, wherein the method does not employ any of the canonical dNTP corresponding to the destabilizing dNTP.

Embodiment 22: The method of any one of embodiments 19-21, wherein the amplification is carried out in multiplex.

Embodiment 23: The method of any one of embodiments 19-22, wherein the amplification is selected from standard polymerase chain reaction (PCR), isothermal amplification, thermal convection PCR, base-3 PCR, base-6 PCR, echo amplification, and krypton amplification.

Embodiment 24: The method of embodiment 23, wherein the method provides one or more of the following features as compared to PCR in the absence of any destabilizing dNTP: faster thermal cycling profiles in standard PCR; faster time-to-result (TTR) in isothermal PCR; faster TTR in thermal convection PCR; and reduced differences in amplification due to differences in G/C content of target nucleic acids.

Embodiment 25: The method of embodiment 24, thermal cycling is carried out with a cycling time in the range of about 5 to about 15 seconds, optionally about 7 to about 13 seconds, about 8 to about 11 seconds, or about 9 to about 10 seconds.

Embodiment 26: The method of embodiment 24 or embodiment 25, wherein thermal cycling is carried out with a difference between annealing and denaturation temperatures of less than about 50 degrees C., optionally less than about 45 degrees C., about 40 degrees C., about 35 degrees C., or about 30 degrees C.

Embodiment 27: The method of any one of embodiments 24-26, wherein the TTR is less than or equal to about 15 minutes, optionally less than about 10 minutes, about 7 minutes, or about 5 minutes.

Embodiment 28: The method of embodiment 27, wherein the method is capable of detecting about 10,000 copies of a human genomic DNA target in about 100 to about 500 seconds, optionally about 250 to about 350 seconds.

Embodiment 29: The kit of any one of embodiments 1-17 or the method of any one of embodiments 19-28, wherein the destabilizing dNTP is selected from 2-deoxyinosine-5-triphosphate (dITP), 7-deaza-2-deoxinosine-5-triphosphate, and N4-methyl dCTP (methyl dCTP).

Embodiment 30: The kit of any one of embodiments 1-17, the method of any one of embodiments 19-28, or the kit or method of embodiment 29, wherein the kit comprises, or the method employs, a component selected from the group consisting of a PCR enhancer, a volume-excluding reagent, and a polymerase faster than Taq polymerase.

Embodiment 31: The kit of any one of embodiments 1-17, the method of any one of embodiments 19-28, or the kit or method of embodiment 29 or embodiment 30, wherein the target nucleic acid is RNA, and the kit comprises or the method employs a reverse transcriptase.

### BRIEF DESCRIPTION OF THE DRAWINGS

FIG. 1: PCR data demonstrating equal performance when dITP is substituted for dGTP in PCR a reaction (Example 1).

FIG. 2: Post-PCR melt demonstrating the reduction in amplicon Tm when dITP is incorporated in place of dGTP (Example 1).

FIG. 3: PCR with lowered denaturation temperature and time. Due to the reduced amplicon Tm, amplification is observed only using dITP as a replacement for dGTP. Time per cycle is ˜15 s including temperature ramp, hold, and optical read (Example 2, Single-plex).

FIG. 4: Simultaneous detection of hgDNA and Chlamydia DNA using fast cycling parameters with dITP (Example 2, Multi-plex-Two Targets).

FIG. 5A-5E: Simultaneous detection of hgDNA, Chlamydia DNA, and Neisseria DNA with dITP (Example 2, Multi-plex-Three Targets at Various Copy Levels). Detection at 10,000 copies/reaction (5A), 2000 copies per reaction (5B), 400 copies/microliter (5C), 80 copies/microliter (5D), and 16 copies/microliter (5E).

FIG. 6: Detection of SARS-COV-2 RNA (RdRP) by RT-PCR, with dITP as a replacement for dGTP (Example 2, Single-plex RT-PCR).

FIG. 7: Isothermal amplification of hgDNA (Example 4). Hold temperature of 65° C. Use of dITP gives a several-minute improvement over standard PCR mix containing dGTP.

FIG. 8: Use of 7-deaza-2-deoxinosine-5-triphosphate (7-deaza-dITP) in PCR yields improved Ct and endpoint fluorescence results, as compared to dGTP and dITP (Example 5).

FIG. 9: Amplicon Tm analysis of amplicons produced from PCR in the presence of 7-deaza-dITP, as compared to dGTP and dITP (Example 5).

FIG. 10: Use of 7-deaza-2-deoxinosine-5-triphosphate (7-deaza-dITP) in PCR yields a ˜30% increase in endpoint fluorescence (Example 5).

FIG. 11A-11B: Schematic representations of designs for convectively-driven thermal cyclers for use in PCR (11A; Wheeler, E. K., et al. (2004) Anal. Chem. 76:4011-4016) and an illustrative embodiment of the methods described herein (11B).

FIGS. 12 and 13: Schematic drawings showing various elements of illustrative embodiments of the chimeric primers described herein. 12 shows looped (“hairpin”) primers, and FIG. 13 shows linear primers. The architecture of the chimeric primers includes two main segments. As shown for both the hairpin and linear versions, the universal adapter sequences (1) can have optional non-natural DNA and ribobases that are non-palindromic to the target sequence and designed to have a T_{M} greater than the reaction temperature. The target-specific sequence (4) (not including the terminal 3′ cap) has a T_{M} greater than the reaction temperature.

FIG. 14: An illustrative summary of the products formed through various isothermal amplification pathways that can exist in echo amplification. Circular arrows depict the linear amplification pathways, while the horizontal arrows shown are the exponential pathways. Amplification of each strand can be controlled independently in the forward direction (solid arrows) and the reverse direction (dashed arrows) through controlling the ratios of different non-natural and natural NTPs and dNTPs, primer mixtures of non-natural ribobases and deoxynucleoside bases, or primer biasing. In one embodiment, utilization of a cycling probe allows continued detection of the forward strand (as shown). In some embodiments, the cycling probe detection is in the reverse direction. Continued probe detection, in either direction, as the amplification pathways progress through the linear and exponential phases is accomplished with the strand that is more abundant.

FIG. 15: Additional possible pathways in echo amplification, whereby the minimal amplification duplex can be restored to hemi-amplification duplexes through additional priming and extension events. Further priming and extension events lead to hemi-amplification duplexes being restored to full amplification duplexes.

FIG. 16: A schematic drawing showing an illustrative embodiment of krypton amplification. This method employs primers with Tms that differ significantly; as shown, the Tm difference between the high-Tm primer and the low-Tm primer is at least 10° C.

FIG. 17: A schematic drawing showing an illustrative temperature/time course of a single cycle of krypton amplification. This drawing shows a single temperature pulse inserted into the denaturation phase at 95° C. Before this pulse, denaturation has separated two strands of target nucleic acid duplex, as shown. During the temperature pulse, the temperature is reduced to an appropriate temperature for the high-Tm primer to bind and produce a new nucleic acid strand. In this illustration, the high-Tm primer is the “forward” primer (“F primer”), which produces a “sense” (or “top” or 5′-to-3′, reading left-to-right) strand, although the method works just as well with the high-Tm primer being the “reverse” primer (“R primer”), in which case this step would produce an “antisense” (or “bottom” or 3′-to-5′, reading left-to-right) strand. In either case, the pulse ends with a return to the denaturation temperature and three nucleic acid strands that can each serve as a template when the temperature is reduced to an appropriate temperature for the low-Tm primer to bind the available templates (here, two sense strands) and produce new nucleic acid strands. At this temperature, the high-Tm primer will also bind the available template (here, the antisense strand) to produce a new nucleic acid strand. As a result, a single cycle of amplification ends with 3 copies of initial, single nucleic acid duplex, resulting in base-3 PCR.

### DETAILED DESCRIPTION

### Definitions

Terms used in the claims and specification are defined as set forth below unless otherwise specified.

The term “nucleic acid” refers to a nucleotide polymer, and unless otherwise limited, includes analogs of natural nucleotides that can function in a similar manner (e.g., hybridize) to naturally occurring nucleotides.

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include, but are not limited to, base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like.

More particularly, in some embodiments, nucleic acids, can include, but are not limited to, polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “complementary” refers to the capacity for precise pairing between two nucleotides; i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands. A base that is complementary to another base is said to “hybridize to” that base.

“Specific hybridization” refers to the binding of a nucleic acid to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

In some embodiments, hybridizations are carried out under stringent hybridization conditions. The phrase “stringent hybridization conditions” generally refers to a temperature in a range from about 5° C. to about 20° C. or about 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), both incorporated herein by reference for their descriptions of stringent hybridization conditions). As indicated by standard references, a simple estimate of the Tₘ value may be calculated by the equation: Tₘ=81.5+0.41 (% G+C), when a nucleic acid is in aqueous solution at 1 M NaCl (see, e.g., Anderson and Young, Quantitative Filter Hybridization in NUCLEIC ACID HYBRIDIZATION (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH7. Tₘ calculation for oligonucleotide sequences based on nearest-neighbors thermodynamics can carried out as described in “A unified view of polymer, dumbbell, and oligonucleotide DNA nearest-neighbor thermodynamics” John SantaLucia, Jr., PNAS Feb. 17, 1998 vol. 95 no. 4 1460-1465 (which is incorporated by reference herein for this description).

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least about 7 nucleotides long and, in some embodiments, range from about 10 to about 30 nucleotides, or, in some embodiments, from about 10 to about 60 nucleotides, in length. In some embodiments, primers can be, e.g., about 15 to about 50 nucleotides long. Short primer molecules generally require cooler temperatures to form sufficiently stable hybrid complexes with the template. A primer need not reflect the exact sequence of the template but must be sufficiently complementary to hybridize with a template.

A primer is said to anneal to another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in some embodiments, amplification primers used herein are said to “anneal to” or be “specific for” a nucleotide sequence.” This description encompasses primers that anneal wholly to the nucleotide sequence, as well as primers that anneal partially to the nucleotide sequence.

The term “primer pair” refers to a set of primers including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. As will be recognized by those of skill in the art, the terms “upstream” and “downstream” or “forward” and “reverse” are not intended to be limiting, but rather provide illustrative orientations in some embodiments.

A “probe” is a nucleic acid capable of binding to a target nucleic acid of complementary sequence through one or more types of chemical bonds, generally through complementary base pairing, usually through hydrogen bond formation, thus forming a duplex structure. The probe can be labeled with a detectable label to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size. Generally, probes are at least about 7 to about 15 nucleotides in length. Other probes are at least about 20, about 30, or about 40 nucleotides long. Still other probes are somewhat longer, being at least about 50, about 60, about 70, about 80, or about 90 nucleotides long. Yet other probes are longer still, and are at least about 100, about 150, about 200 or more nucleotides long. Probes can also be of any length that is within any range bounded by any of the above values (e.g., about 15 to about 20 nucleotides in length).

The primer or probe can be perfectly complementary to the target nucleotide sequence or can be less than perfectly complementary. In some embodiments, the primer has at least about 65% identity to the complement of the target nucleotide sequence over a sequence of at least about 7 nucleotides, more typically over a sequence in the range of about 10 to about 30 nucleotides, and, in some embodiments, over a sequence of at least about 14 to about 25 nucleotides, and, in some embodiments, has at least about 75% identity, at least about 85% identity, at least about 90% identity, or at least about 95%, about 96%, about 97%, about 98%, or about 99% identity. It will be understood that certain bases (e.g., the 3′ base of a primer) are generally desirably perfectly complementary to corresponding bases of the target nucleotide sequence. Primer and probes typically anneal to the target sequence under stringent hybridization conditions.

As used herein with reference to a portion of a primer or a nucleotide sequence within the primer, the term “specific for” a nucleic acid, refers to a primer or nucleotide sequence that can specifically anneal to the target nucleic acid under suitable annealing conditions.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include standard, thermocycling-based PCR (employing a “forward” and “reverse” primer that are complementary to primer binding sites in the target nucleic acid sequence), nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), helicase-dependent amplification (HDA), and the like. Descriptions of such techniques can be found in, among other sources, Ausubel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4 (1): 41-7, U.S. Pat. Nos. 6,027,998; 6,605,451, Barany et al., PCT Publication No. WO 97/31256; Wenz et al., PCT Publication No. WO 01/112579; Day et al., Genomics, 29 (1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27: e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13 (2): 294-307, and Landegren et al., Science 241:1077-80 (1988), Demidov, V., Expert Rev Mol Diagn. 2002 November; 2 (6): 542-8., Cook et al., J Microbiol Methods. 2003 May; 53 (2): 165-74, Schweitzer et al., Curr Opin Biotechnol. 2001 February; 12 (1): 21-7, U.S. Pat. Nos. 5,830,711, 6,027,889, 5,686,243, PCT Publication No. WO0056927A3, and PCT Publication No. WO9803673A1. Also encompassed by the term “amplification” are isothermal amplification, thermal convection PCR, base-3 PCR, base-6 PCR, echo amplification, and krypton amplification, which are described below.

A “multiplex amplification reaction” is one in which two or more nucleic acids distinguishable by sequence are amplified simultaneously.

The term “qPCR” is used herein to refer to quantitative real-time polymerase chain reaction (PCR), which is also known as “real-time PCR” or “kinetic polymerase chain reaction;” all terms refer to PCR with real-time signal detection.

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation.

The naturally occurring bases adenine, thymine, uracil, guanine, and cytosine, which make up DNA and RNA, are described herein as “canonical” or “unmodified.”

The term “modified base” is used herein to refer to a base that is not a canonical base (e.g., adenine, thymine, uracil, guanine, and cytosine).

Nucleotides comprising modified bases are referred to herein as “modified nucleotides.”

Common modifications to canonical bases can make them “stabilizing” or “destabilizing.” These terms refer to a greater or lesser tendency, respectively, for the modified base to pair an unmodified complementary base, as compared to the tendency of canonical bases to form base pairs (e.g., A-T and G-C). Stabilizing and destabilizing bases can exert their effects by differences in, e.g., hydrogen bonding in base pairing, in base stacking, and effects on the secondary structure of sequences in which they appear. Stabilizing and destabilizing bases are known; some are known to exist in nature (e.g., inosine), and some have been produced by synthetically modifying a canonical base.

A base and a deoxynucleotide triphosphate (dNTP) are said to “correspond” if the if the dNTP comprises the base.

As used herein, the “Tm” of an oligonucleotide, such as a primer or probe, refers to the Tm (melting temperature) of a double-stranded form of the oligonucleotide.

All ranges described herein include their endpoints, unless otherwise indicated.

The notation “\hat{ }X” refers to the base for exponential nucleic acid amplification. Classical PCR is base 2 and therefore denoted as “PCR\hat{ }2.” PCR\hat{ }3 refers to base 3 PCR, PCR\hat{ }4 reference to base 4 PCR, and so on.

Use of Destabilizing Bases to Reduce Amplicon Tm and/or Secondary Structure

The present disclosure describes the use of modified deoxynucleotide triphosphates (dNTPs) in a nucleic acid amplification reaction mixture to improve the efficiency of the amplicon denaturing or stand-separation step during amplification and increase the flexibility of single-stranded amplicon folding/coiled structure to favor primer and optional probe hybridization. Specifically, the use of destabilizing dNTPs allows for one or more of the following: 1) fast thermal cycling profiles in standard polymerase chain reaction (PCR), 2) improved time-to-response (TTR) in isothermal PCR, 3) improved TTR when using thermal convection PCR (i.e., gradient), 4) balanced thermal amplification conditions between A/T and G/C rich sequences, and 5) reduced amplicon folding structures that can enhance the efficiency of primer annealing.

This approach has been demonstrated in a model system to detect a specific human genomic DNA (hgDNA) sequence using deoxyinosine triphosphate (dITP) in a PCR master mix. This method required about 300 seconds (5-6 min) to detect about 10,000 copies of target. The equivalent system using standard dNTPs does not amplify target sequences. Fast thermal cycling profiles were achieved by using dITP to reduce the temperature difference between anneal and denaturing reactions in PCR and/or facilitate strand displacement without special enzymes. This approach also provides greater flexibility for the location of a target sequence for amplification (i.e., by balancing out A/T and G/C rich sequence requirements) and improves multiplexing, especially for fast TTR applications. The of dITP has enabled fast and efficient PCR or RT-PCR assays capable of automated detection of multiple targets in under 15 minutes total acquisition time (including sample preparation) in our model system. The technology has enabled standard PCR in less than 7 minutes.

This approach does not require: 1) complex and expensive new chemistries, 2) complex oligonucleotide designs, 3) complex PCR reaction schemes, 4) custom, proprietary, and/or expensive enzymes, 5) new hardware for automation, or 6) laborious product development or prolonged time to design lock and/or time to market. Instead, this approach depends on thermally balancing primers, optional probes, and amplicons produced upon amplification to ensure a level of incorporation of destabilizing bases into an amplicon to lower amplicon Tₘ, without unduly reducing the ability of primers and probes to anneal to individual amplicon strands for subsequent rounds of amplification.

### Primer and Probe Design

### General Considerations

Primers suitable for nucleic acid amplification are sufficiently long to prime the synthesis of extension products in the presence of a suitable nucleic acid polymerase. The exact length and composition of the primer will depend on many factors, including, for example, temperature of the annealing reaction, source and composition of the primer, and where a probe is employed, proximity of the probe annealing site to the primer annealing site and ratio of primer:probe concentration. For example, depending on the complexity of the target nucleic acid sequence, an oligonucleotide primer typically contains in the range of about 10 to about 60 nucleotides, although it may contain more or fewer nucleotides. The primers should be sufficiently complementary to selectively anneal to their respective strands and form stable duplexes.

In general, one skilled in the art knows how to design suitable primers capable of amplifying a target nucleic acid of interest. For example, PCR primers can be designed by using any commercially available software or open-source software, such as Primer3 (see, e.g., Rozen and Skaletsky (2000) Meth. Mol. Biol., 132:365-386; www.broad.mit.edu/node/1060, and the like) or by accessing the Roche UPL website. The amplicon sequences are input into the Primer3 program with the UPL probe sequences in brackets to ensure that the Primer3 program will design primers on either side of the bracketed probe sequence.

Primers may be prepared by any suitable method, including, for example, direct chemical synthesis by methods such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetra. Lett., 22:1859-1862; the solid support method of U.S. Pat. No. 4,458,066 and the like or can be provided from a commercial source. Primers may be purified by using a Sephadex column (Amersham Biosciences, Inc., Piscataway, NJ) or other methods known to those skilled in the art. Primer purification may improve the sensitivity of the methods described herein.

### Thermal Balancing of Primers, Probe, and Amplicon

The stability of a nucleic acid duplex can be modified by adjusting the length of a sequence, the G-C content, and/or by including stabilizing or destabilizing base(s) in the sequence. The Tm of primers and probes can be adjusted by selecting target sequences of appropriate length and composition and through the inclusion of appropriate modified bases in the primer/probe sequence. The Tm of amplicons can be adjusted by selecting amplicons of appropriate length and composition, taking into account that one or more destabilizing dNTPs in the amplification reaction mix are incorporated into the amplicon strands during amplification. When a newly synthesized strand becomes available for primer/probe hybridization, that strand may have incorporated one or more destabilizing bases within the primer/probe target sequences. A primer or probe is said to “face” a destabilizing base if the destabilizing base in present in the target sequence to which the primer or probe hybridizes.

In some embodiments, primers, probes, and amplicons can be selected and designed so that no destabilizing bases are incorporated into the target sequences for the primers or probes. Amplicons and primer/probe target sequences can be selected so that the amplicon sequence will incorporate some destabilizing bases to lower amplicon Tm, but these bases are located outside of primer/probe target sequences.

In some embodiments, it may not be possible to select/design amplicons, primers, and probes that completely avoid the incorporation of destabilizing bases into primer/probe target sequences. In such cases, primers/probes may face one or multiple destabilizing bases, which has the effect of destabilizing primer/probe hybridization. This destabilizing effect is, in some embodiments, countered by designing primers/probes to include one or multiple stabilizing bases.

The present method entails using or more destabilizing dNTP in the amplification reaction mixture to reduce amplicon Tm and/or secondary structure, while selecting/designing amplicons, primers, and optional probes that are thermally balanced to provide one or more of the advantages outlined above, as compared to standard PCR.

Stabilizing modifications for primers and probes include, e.g., stretches of peptide nucleic acids (PNAs) that can be incorporated into DNA oligonucleotides to increase duplex stability. Locked nucleic acids (LNAs) and unlocked nucleic acids (UNAs) are analogues of RNA that can be easily incorporated into DNA oligonucleotides during solid-phase oligonucleotide synthesis, and respectively increase and decrease duplex stability. Other examples useful, in some embodiments, for probes include peptide nucleic acids (PNAs) and minor groove binders (MGBs) for probes. Suitable stabilizing bases also include modified DNA bases that increase the stability of base pairs (and therefore the duplex as a whole). These modified bases can be incorporated into oligonucleotides during solid-phase synthesis and offer a more predictable method of increasing DNA duplex stability. Examples include, but are not limited to, AP-dC (G-clamp), SUPER A/T and ppG (both available from ELITech), 2,6-diaminopurine (2-aminoadenine), C-5 propynyl-dC (pdC) for dC, C-5 propynyl-dU (pdU) for dT, 5-methyl-dC, LNAs. Cepheid employs proprietary stabilizing versions of A, T, G, and C in its commercial assays.

“Destabilizing bases” are those that destabilize double-stranded DNA by virtue of forming less stable base pairs than the typical A-T and/or G-C base pairs.

Inosine (I) is a destabilizing base because it pairs with cytosine (C), but an I-C base pair is less stable than a G-C base pair. This lower stability results from the fact that inosine is a purine that can make only two hydrogen bonds, compared to the three hydrogen bonds of a G-C base pair. “Destabilizing nucleotide triphosphates (dNTPs)” are the triphosphate forms of destabilizing bases. Examples of the latter include, but are not limited to, 2-deoxyinosine-5-triphosphate (dITP), 7-deaza-2-deoxinosine-5-triphosphate, and N4-methyl dCTP. Other destabilizing dNTPs are known to, or readily identified by, those of skill in the art.

Thermal balancing of primers, probes and amplicons can be carried using, e.g., bioinformatics, in silico testing, and empirical studies. In some embodiments, bioinformatics can be used to assist with selecting the amplicon, and more specifically, the location of the primers and probes. This approach can be used to ensure that the number of destabilizing bases, after amplification, facing the primers are minimized, with, e.g., zero, two, or one destabilizing chemistries facing the primer, if possible. If the bioinformatics does not allow for this arrangement, then stabilization of the primers and/or probes can be carried out to balance or overcome the effect of the destabilizing bases. Stabilization of the primers and probes may not be required for optimal performance, depending on the specific sequences, primer/probe Tms and numbers of destabilizing mods in complementary strands after amplification. In silico and/or empirical studies can easily be carried out to assess the effects of different types and/or amounts of stabilization. Example 1, below, describes an illustrative approach to designing thermally balanced primers, probe, and amplicon for particular circumstances.

### Samples

Nucleic acid-containing samples can be obtained from biological sources and prepared using conventional methods known in the art. In particular, nucleic acid samples useful in the methods described herein can be obtained from any source, including unicellular organisms and higher organisms such as plants or non-human animals, e.g., canines, felines, equines, primates, and other non-human mammals, as well as humans. In some embodiments, samples may be obtained from an individual suspected of being, or known to be, infected with a pathogen (e.g., viral, bacterial, fungal or parasitic), an individual suspected of having, or known to have, a disease, such as cancer, or a pregnant individual.

Nucleic acids can be obtained from cells, bodily fluids (e.g., blood, a blood fraction, urine, etc.), or tissue samples by any of a variety of standard techniques. In some embodiments, the method employs samples of plasma, serum, spinal fluid, lymph fluid, peritoneal fluid, pleural fluid, oral fluid, and external sections of the skin; samples from the respiratory, intestinal genital, or urinary tracts; samples of tears, saliva, blood cells, stem cells, or tumors. Samples can be obtained from live or dead organisms or from in vitro cultures. Illustrative samples can include single cells, paraffin-embedded tissue samples, and needle biopsies. In some embodiments, the nucleic acids analyzed are obtained from a single cell.

Nucleic acids of interest can be isolated using methods well known in the art. The sample nucleic acids need not be in pure form but are typically sufficiently pure to allow the steps of the methods described herein to be performed.

### Target Nucleic Acids

Any target nucleic acid that can detected by nucleic acid amplification can be detected using the methods described herein. In typical embodiments, at least some nucleotide sequence information will be known for the target nucleic acids. For example, if the amplification reaction employed is PCR, sufficient sequence information is generally available for each end of a given target nucleic acid to permit design of suitable amplification primers.

The targets can include, for example, nucleic acids associated with pathogens, such as viruses, bacteria, protozoa, or fungi; RNAs, e.g., those for which over- or under-expression is indicative of disease, those that are expressed in a tissue- or developmental-specific manner; or those that are induced by particular stimuli;

genomic DNA, which can be analyzed for specific polymorphisms (such as SNPs), alleles, or haplotypes, e.g., in genotyping. Of particular interest are genomic DNAs that are altered (e.g., amplified, deleted, and/or mutated) in genetic diseases or other pathologies; sequences that are associated with desirable or undesirable traits; and/or sequences that uniquely identify an individual (e.g., in forensic or paternity determinations).

### Polymerase

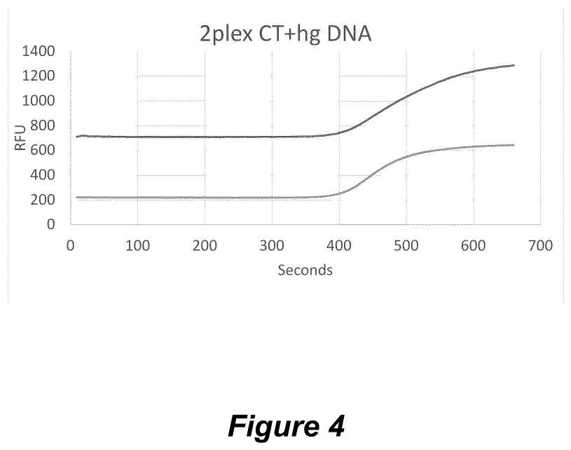

The disclosed methods make use of a polymerase for amplification. Such polymerases are well known and those skilled in the art can readily select a suitable polymerase for a particular embodiment. In some embodiments, the polymerase is a DNA polymerase that either has or lacks a 5′ to 3′ exonuclease activity. In some embodiments, the polymerase is one that is adapted for a “hot start.” The most widely used polymerases are natural and mutant forms of Taq and Tth polymerases. Suitable polymerases include, but are not limited to, APTATAQ, KAPAG, PHUSION, PRIMESTAR, AMPLITAQ, GOTAQ, EXTAQ, PLATINUM, HERCULASE II, KLENTAQ, TITANIUM, KAPA2G, KAPA3G, PAQ5000, KOD, and SPEEDSTAR. Other polymerases useful in the methods described herein are well known and include, for example, those shown in FIG. 4 of Montgomery et al. (2013) Analytical Biochemistry, 44 (2): 133-139, which is hereby incorporated by reference herein for this description.

Illustrative polymerase concentrations range from about 10 to about 200 units per reaction. In various embodiments, the polymerase concentration can be at least about: 10, 20, 30, 40, 50, 60, 70, 80, 90, 100, 110, 120, 130, 140, 150, 160, 170, 180, 190, or 200 or more units per reaction. In some embodiments, the polymerase concentration falls within a range bounded by any of these values, e.g., about 10 to about 200, about 10 to about 150, about 10 to about 100, about 10 to about 50, about 20 to about 150, about 20 to about 100, about 20 to about 50, about 50 to about 200, about 50 to about 150, about 50 to about 100, about 100 to about 200, about 100 to about 150, etc. units per reaction.

### Reverse Transcriptase

The disclosed methods can make use of a reverse transcriptase, for example, when the target nucleic acids are RNA. Such reverse transcriptases are well known and those skilled in the art can readily select a suitable enzyme for a particular embodiment. Suitable reverse transcriptases include, but are not limited to, M-MLV, Superscript II-IV, Luna, EnzScript, NxtScript, NxtScript 2G, and GoScript.

Illustrative reverse transcriptase concentrations range from about 10 to about 200 units per reaction. In various embodiments, the reverse transcriptase concentration can be at least about: 10, 20, 30, 40, 50, 60, 70, 80, 90, 100, 110, 120, 130, 140, 150, 160, 170, 180, 190, or 200 or more units per reaction. In some embodiments, the polymerase concentration falls within a range bounded by any of these values, e.g., about 10 to about 200, about 10 to about 150, about 10 to about 100, about 10 to about 50, about 20 to about 150, about 20 to about 100, about 20 to about 50, about 50 to about 200, about 50 to about 150, about 50 to about 100, about 100 to about 200, about 100 to about 150, etc. units per reaction.

### Amplification

The primer sets described above are contacted with sample nucleic acids under conditions wherein the primers anneal to their template strands, if present. The desired nucleic acid amplification method is carried out using a suitable DNA polymerase. Various nucleic acid amplification methods are known and can be employed in the methods described herein.

In some embodiments, the amplification step is performed using PCR. For running real-time PCR reactions, reaction mixtures generally contain an appropriate buffer, a source of magnesium ions (Mg²⁺) in the range of about 1 to about 10 mM, e.g., in the range of about 2 to about 8 mM, nucleotides, and optionally, detergents, and stabilizers. An example of one suitable buffer is TRIS buffer at a concentration of about 5 mM to about 85 mM, with a concentration of about 10 mM to about 30 mM being preferred. In one embodiment, the Tris buffer concentration is about 20 mM in the reaction mix double-strength (2×) form. The reaction mix can have a pH range of from about 7.5 to about 9.0, with a pH range of about 8.0 to about 8.5 as typical. Concentration of nucleotides can be in the range of about 25 mM to about 1000 mM, typically in the range of about 100 mM to about 800 mM. Examples of dNTP concentrations include about: 100, 200, 300, 400, 500, 600, 700, and 800 mM. A non-limiting list of detergents such as TWEEN 20, TRITON X 100, BRIJ and NONIDET P40 may also be included in the reaction mixture. Stabilizing agents such as dithiothreitol (DTT, Cleland's reagent) or mercaptoethanol may also be included. In addition, master mixes may optionally contain dUTP as well as uracil DNA glycosylase (uracil-N-glycosylase, UNG). A master mix is commercially available from Applied Biosystems, Foster City, CA, (TaqMan® Universal Master Mix, cat. nos. 4304437, 4318157, and 4326708). In some embodiments, the reaction mixture contains one or more amplification enhancers, such as, e.g., dimethyl sulfoxide (DMSO), bovine serum albumin (BSA), tetramethylsulfoxide (TMSO), acetamide, betaine, and the like. DMSO is a Tm-reducing agent and can be used to reduce amplicon Tm by about 3-4 degrees; consequently, the time required for the denaturation step can be reduced. Amplification reactions may also include an aptamer or antibody to facilitate “hot start.”

Those skilled in the art can select appropriate thermal cycling profiles for a given application. Two illustrative thermal profile approaches yield a fast time-to-result (TTR) due to low cycle times. In “fast” toggling, one waits for temperature equilibration between denaturation and annealing temperatures, with the equilibration time not included in the “hold” time at a particular temperature. An exemplary fast toggling profile is: about 95° C. for about 10 seconds, about 58° C. for about 10 seconds, followed by cycles of about 77° C. for about 1 second and about 58° C. for about 1 second. For such a profile, each cycle can take about 15 seconds. In “overheat” toggling the is no time for equilibration between temperature changes. The denaturation temperature is set higher than in fast toggling, and since no time is allowed for the reaction to reach that temperature, the actual temperature reached by the reaction during this phase is unknown. An exemplary overheat toggling profile is: about 95° C. for about 10 seconds, about 58° C. for about 10 seconds, followed by cycles of about 96° C. for about 2 seconds and about 58° C. for about 5 seconds. For such a profile, each cycle can take about 8 seconds.

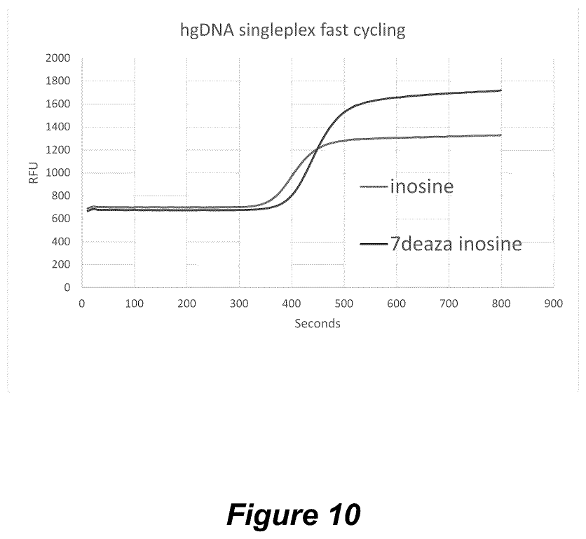

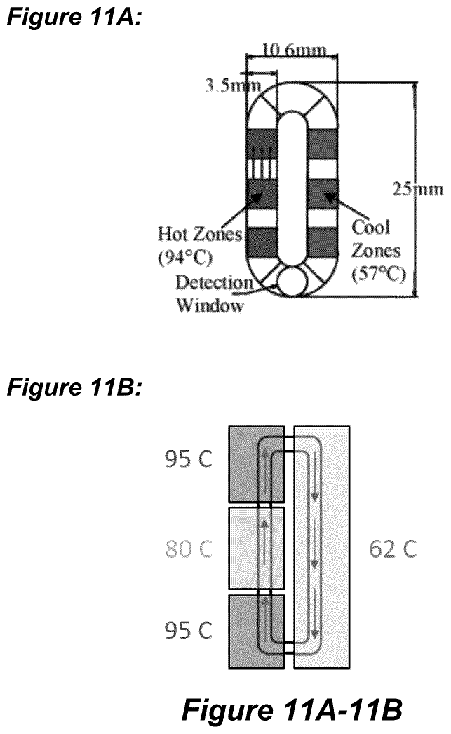

Other illustrative nucleic acid amplification methods useful in the methods described herein include isothermal amplification, thermal convection PCR (see FIGS. 10A and 10B), base-3 PCR, base-6 PCR, “echo amplification,” and “krypton amplification.” Base-3 PCR entails using a primer set including three oligonucleotides at one end of an amplicon. Base-6 PCR entails using a primer set including three oligonucleotides at both ends of an amplicon. Base-3 and -6 PCR are described in U.S. Pat. No. 11,352,622 (which is hereby incorporated by reference herein for this description), PCT Publication No. WO 2021/222289 (which is hereby incorporated by reference herein for this description), and PCT Publication No. 2021/222144 (which is hereby incorporated by reference herein for this description).

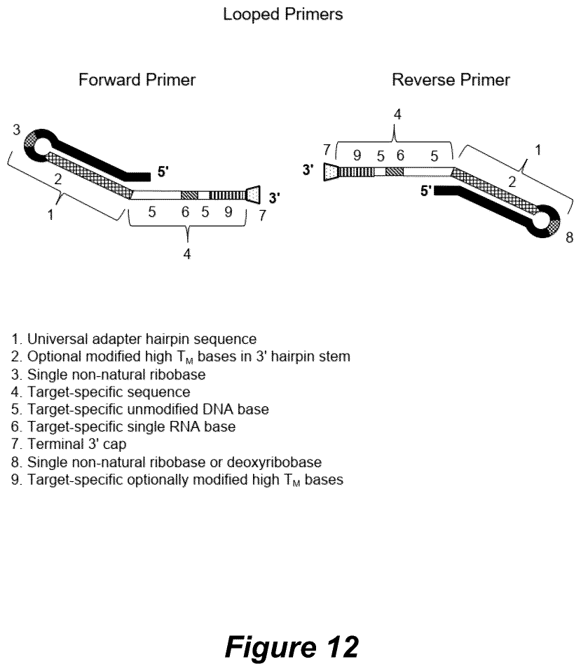

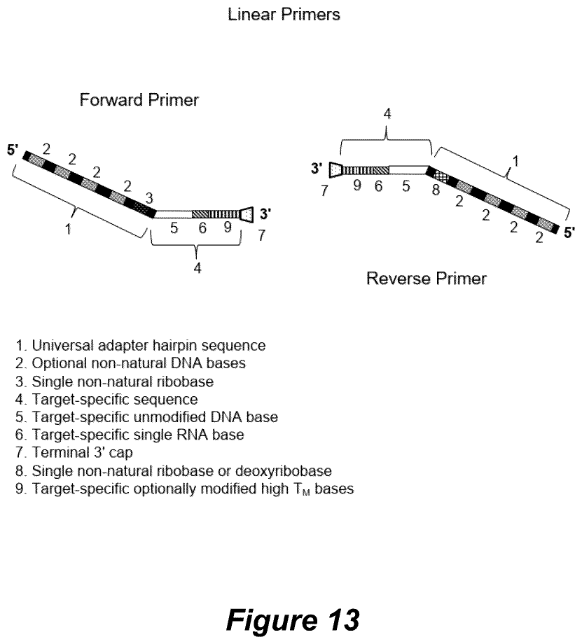

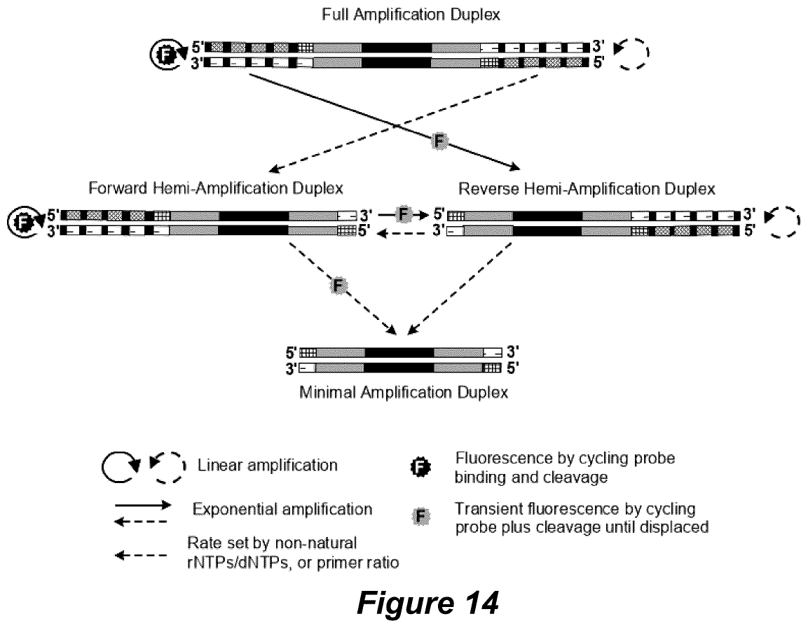

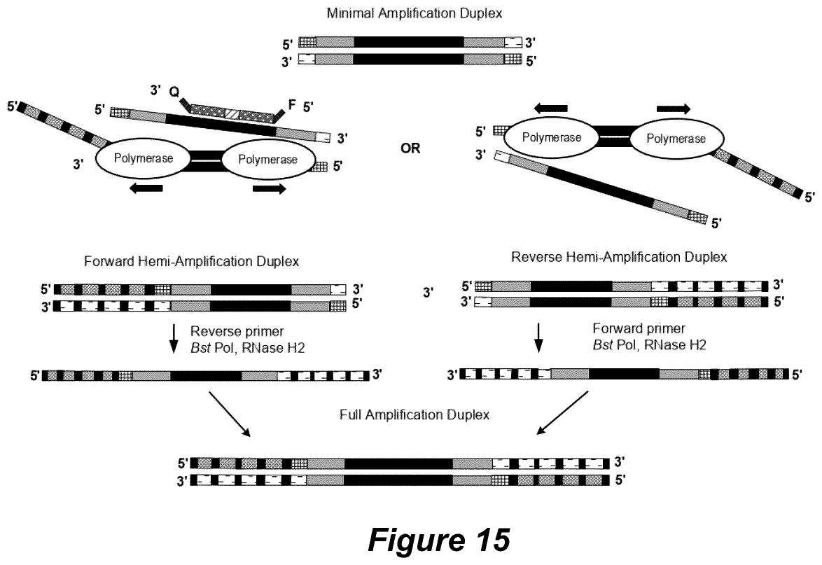

Echo amplification is so-named because of the effect of including a cleavable site located in each primer—including an RNA ribobase in an illustrative embodiment—that is only activated after the complementary strand is fully synthesized and only then initiates a second return of the strand synthesis along the nascent complementary strand, evocative of an echo reverberation off of a surface. In some embodiments, primers remain inactive unless and until they are hybridized to a specific target sequence. In some embodiments, primers incorporate universal adapters that then direct linear amplification of each strand independently. The linear amplification of each strand can then act as the template for the opposing primer, thus triggering an exponential amplification cycle. In some embodiments, each primer encodes the information signal to initiate a first-strand synthesis, and then also a return synthesis on subsequent rounds of amplification. One of shortest and simplest methods for achieving this is to include a ribonucleotide in the primer that when bound to the exact matching DNA sequence (i.e., when there is an exact match between the ribonucleotide and deoxynucleotide complement), then becomes the substrate for the excision repair pathway of RNase H2. After excision, RNase H2 leaves a free 3′ hydroxyl group on the primer fragment that was 5′ of the ribobase, which can then be extended by a DNA-dependent DNA polymerase with strand displacement activity. Though not required, with the use of a chimeric fusion enzyme that has both functions in different domains of the same enzyme, this process can proceed with both steps in rapid succession. Then, there can also be a second ribobase, a non-natural ribobase as depicted in FIG. 12, that is initially held in an inactive state, for example, by locating this second, non-natural ribobase it unpaired in a small loop structure at the 5′ end of the primer. This second, non-natural ribobase in the loop structure is further protected because it is not composed of a naturally occurring base, so it is unlikely to ever come across its exact complement in a sample by chance. In some embodiments the primer incorporates a DNA sequence on the 5′ end that is not known to exist in any organism that might be present in a clinical sample, and thus that region would not bind to form an active site for cleavage even without a loop structure. This effect can be further enhanced by incorporating other non-natural DNA bases in this region to further differentiate the sequence from any naturally occurring nucleic acid sequences, as shown in FIG. 13. While the structures of the forward and reverse primers are conceptually similar, in the first embodiment each has a different ribobase in the loop region that is formed from different non-natural xenobiotic bases that do not have any complementarity to the standard A, C, G, T or U bases. Instead, they have their own base pair, with the complement nucleotide triphosphate provided in the reaction mixture. This design allows for the separate, independent control of forward and reverse strand amplification, minimizing wasted side reactions of cleaving just the very 3′ end of the amplicon off. Further, it also allows for the adjustment of the rate of ongoing linear amplification of each strand independently as well, through the control of nucleotide triphosphates versus deoxynucleotide triphosphates for the specific non-natural bases. FIGS. 13 and 14 illustrate the various possible amplification pathways in echo amplification. An echo amplification system is described in co-owned U.S. Patent Application No. 63/245,149 (which is incorporated by reference herein for this description). The echo amplification approach is applicable to a variety of amplification techniques but is particularly useful for multiplexed isothermal amplification.

Krypton amplification makes use of nucleic acid primer sets that include one or more pairs of high- and low-Tm primers for each target nucleic acid to be amplified. This approach is diametrically opposed to that of standard PCR, for example, in which primers are designed to have Tms that are as close as possible.

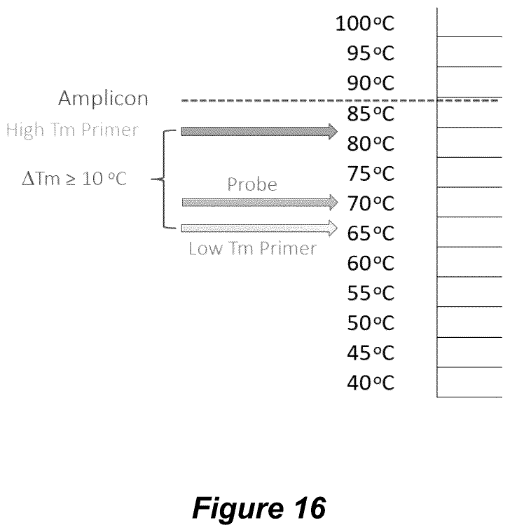

Krypton amplification employs pairs of primers with significantly different Tms, as shown schematically in FIG. 16. In this illustrative embodiment, the high-Tm primer has a Tm at about 82° C., whereas the low-Tm primer has a Tm of just above 65° C. Unlike in standard PCR, the Tm of the probe, in this illustration, in in between that of the high-Tm primer and the low-Tm primer. In various embodiments, the Tm of the probe is at least about 1, 2, 3, 4, 5, 6, 7, 8, 9, or 10 or more degrees C. higher than the Tm of the low-Tm primer. In some embodiments the Tm difference can fall within a range of Tms bounded by any of these values (and including the endpoints). For example, the Tm difference can be at least about: 1-10, 2-9, 3-8, 4-7, or 5-6, etc.

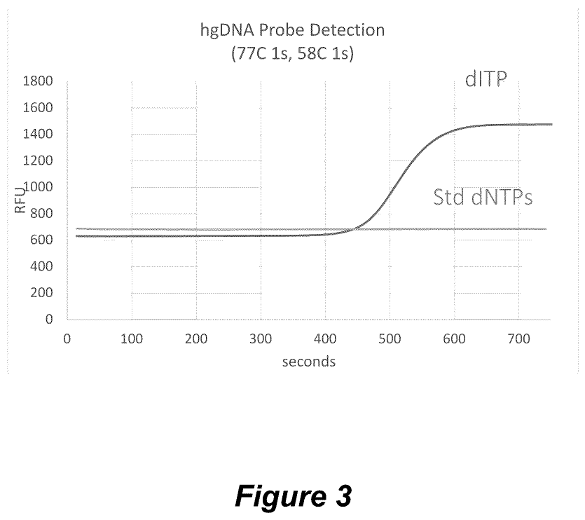

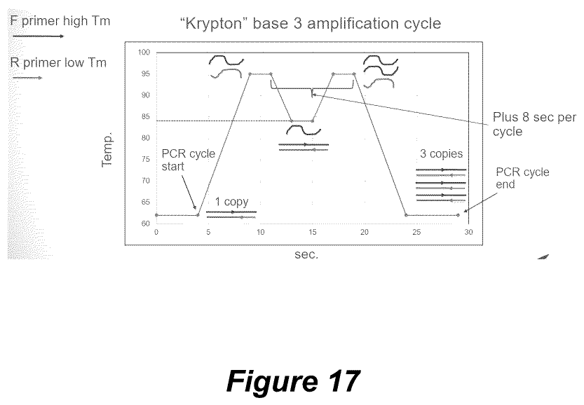

FIG. 17 shows an illustrative temperature/time course of a single cycle of one embodiment of the method described herein, in which the high-Tm primer is the forward primer and the low-Tm primer is the reverse primer. A novel feature of the krypton amplification method is the temperature pulse shown as a dip (which could also be a down-spike) in the center of the temperature/time course of FIG. 16. This temperature pulse is a feature of the present method, regardless of whether the high-Tm primer is the forward primer or the reverse primer. As shown in FIG. 16, the pulse facilitates the synthesis of a third nucleic acid strand from a starting double-stranded nucleic acid duplex. If the high-Tm primer is the forward primer, that third nucleic acid strand will be a sense strand, as shown in FIG. 3. If the high-Tm primer is the reverse primer, that third nucleic acid strand will be an antisense strand. In either case, the pulse ends with a return to the denaturation temperature and three nucleic acid strands that can each serve as a template when the temperature is reduced to an appropriate temperature for the low-Tm primer to bind the available templates and produce new nucleic acid strands. At this temperature, the high-Tm primer will also bind the available template to produce a new nucleic acid strand. As a result, a single cycle of amplification ends with 3 copies of initial, single nucleic acid duplex, resulting in base-3 PCR. In some embodiments, the method is carried out with 2, 3, or 4 more pulses, with the potential to increase the base for exponential amplification to base 4, base 5, or base 6, respectively. Krypton amplification is described in co-owned U.S. Patent Application No. 63/245,149 (which is incorporated by reference herein for this description).

### Labeling Strategies

Any suitable labeling strategy can be employed in the methods described herein. Where the reaction is analyzed for presence of a single amplification product, a universal detection probe can be employed in the amplification mixture. In particular embodiments, real-time PCR detection can be carried out using a universal qPCR probe. Suitable universal qPCR probes include double-stranded DNA-binding dyes, such as SYBR Green, Pico Green (Molecular Probes, Inc., Eugene, OR), Eva Green (Biotium), ethidium bromide, and the like (see Zhu et al., 1994, Anal. Chem. 66:1941-48).

In some embodiments, one or more target-specific qPCR probes (i.e., specific for a target nucleotide sequence to be detected) is employed in the amplification mixtures to detect amplification products. By judicious choice of labels, analyses can be conducted in which the different labels are excited and/or detected at different wavelengths in a single reaction (“multiplex detection”). See, e.g., Fluorescence Spectroscopy (Pesce et al., Eds.) Marcel Dekker, New York, (1971); White et al., Fluorescence Analysis: A Practical Approach, Marcel Dekker, New York, (1970); Berlman, Handbook of Fluorescence Spectra of Aromatic Molecules, 2nd ed., Academic Press, New York, (1971); Griffiths, Colour and Constitution of Organic Molecules, Academic Press, New York, (1976); Indicators (Bishop, Ed.). Pergamon Press, Oxford, 19723; and Haugland, Handbook of Fluorescent Probes and Research Chemicals, Molecular Probes, Eugene (1992); and Linck et al. (2017) “A multiplex TaqMan qPCR assay for sensitive and rapid detection of phytoplasmas infecting Rubus species,” PLOS One 12 (5).

In some embodiments, it may be convenient to include labels on one or more of the primers employed in an amplification mixture.

### Cycling Probes

In some embodiments, a cycling probe can be used for detection and, optionally, quantification of target nucleic acids in the methods described herein. Cycling probes have been used for years as a way of amplifying signal in amplification assays. Cycling probes are described in, e.g., PCT Publication No. WO 89/09284, and U.S. Pat. Nos. 5,011,769 and 4,876,187, which are incorporated herein by reference for this description.

U.S. Pat. No. 5,763,181 describes the use of fluorescently labeled cycling probes to detect target nucleic acids. Generally, the disclosed method employs a fluorescently labeled oligonucleotide substrate containing a nucleotide sequence that is recognized by the enzyme that catalyzes the cleavage reaction. The oligonucleotide substrate can be DNA or RNA and can be single- or double-stranded. The oligonucleotide can be labeled with a single fluorescent label or with a fluorescent pair (donor and acceptor) on a single strand of DNA or RNA. The choice of single- or double-label can depend on the efficiency of the enzyme employed in the method of the invention. There is no limitation on the length of the oligonucleotide substrate, so long as the fluorescent probe is labeled sufficiently far (e.g., 6-7 nucleotides) away from the enzyme cleavage site. Examples of fluorophores commonly used in this method include fluorescein isothiocyanate, fluorescein amine, eosin, rhodamine, dansyl, and umbelliferone. Other fluorescent labels will be known to the skilled artisan. Some general guidance for designing sensitive fluorescently labeled polynucleotide probes can be found in Heller and Jablonski's U.S. Pat. No. 4,996,143. This patent discusses parameters that can be considered when designing fluorescent probes. The cycling probe cleavage reaction can be catalyzed by such enzymes as DNases, RNases, helicases, exonucleases, restriction endonucleases, or retroviral integrases. Other enzymes that effect nucleic acid cleavage are known to the skilled artisan and can be employed to cleave cycling probes having their cognate cleavage sites.

In some embodiments, one or more modified bases can be included in any of the probes described herein. The considerations discussed above regarding the use of stabilizing and/or modified bases in probes also applies to probes.

In some embodiments, it may be convenient to include labels on one or more of the primers employed in in amplification mixture.

### Primer/Probe Concentrations

Primers and, if present, probes can be included in the amplification mixture at concentrations typical for the amplification method (e.g., PCR). In some embodiments, it is advantageous to include the primers at a relatively high concentration to encourage fast annealing/hybridization to facilitate rapid pulse and cycling. In some embodiments, the concentrations of the high-Tm and low-Tm primers are the same, at, for example, at least about 500, 550, 600, 650, 700, 750, 800, or 850 nM (for each primer). In some embodiments, the concentration of each primer fall within a range bounded by any of these values (including the endpoints), e.g., about 550 to about 850 nm, about 600 to about 850 nm, about 650 to about 850 nM, about 700 to about 850 nm, or about 750 to about 850 nm. In the Examples below, the primer concentrations were about 800 nM.

### Exemplary Automation and Systems

In some embodiments, a target nucleic acid is detected using an automated sample handling and/or analysis platform. In some embodiments, commercially available automated analysis platforms are utilized. For example, in some embodiments, the GeneXpert® system (Cepheid, Sunnyvale, CA) is utilized.

The methods described herein are illustrated for use with the GeneXpert® system. Exemplary sample preparation and analysis methods are described below. However, the present invention is not limited to a particular detection method or analysis platform. One of skill in the art recognizes that any number of platforms and methods may be utilized.

The GeneXpert® utilizes a self-contained, single use cartridge. Sample extraction, amplification, and detection may all be carried out within this self-contained “laboratory in a cartridge” (available from Cepheid-see www.cepheid.com). One of skill in the art will recognize that the methods disclosed herein are suitable for use with other cartridge-based systems comprising a cartridge having a plurality of fluidly connected chambers housed within a single disposable cartridge body that provides for automated sample preparation, nucleic acid extraction, amplification, and detection. In some embodiments, the cartridge allows for storage of dried reagents and provides for automated fluidic movements, reagent rehydration, and mixing at the time of sample processing. In some embodiments, the cartridge comprises multiple fluidic pathways to prevent or limit bubble trapping and contamination, while allowing for thermal cycling and optical monitoring of reaction progress in a reaction chamber that extends from the body of the cartridge.

Components of the cartridge include, but are not limited to, processing chambers containing reagents, filters, and capture technologies useful to extract, purify, and amplify target nucleic acids. In some embodiments, a rotary valve enables fluid transfer from chamber to chamber and contains nucleic acids lysis and filtration components. An optical window enables real-time optical detection. A reaction tube enables very rapid thermal cycling.

In some embodiments, the GeneXpert® system includes a plurality of modules for scalability. Each module is configured with fluid sample handling and analysis components.

After the sample is added to the cartridge, the sample is contacted with lysis buffer and released nucleic acid is bound to a nucleic acid-binding substrate such as a silica or glass substrate. The sample supernatant is then removed and the nucleic acid eluted in an elution buffer such as a Tris/EDTA buffer. The eluate may then be processed in the cartridge to detect target genes as described herein. In some embodiments, the eluate is used to reconstitute at least some of the reagents, which are present in the cartridge as lyophilized particles.

In some embodiments, PCR is used to amplify and detect the presence of one or more target nucleic acids. In some embodiments, the PCR uses Taq polymerase with hot start function, such as AptaTaq (Roche).

In some embodiments, an off-line centrifugation is used to improve assay results with samples with low cellular content. The sample, with or without the buffer added, is centrifuged and the supernatant removed. The pellet is then resuspended in a smaller volume of supernatant, buffer, or other liquid. The resuspended pellet is then added to a GeneXpert® cartridge as previously described.

In some embodiments, a continuous, convection flow device can be employed in the method described herein. This device moves the reaction mixture through different temperature zones to achieve thermocycling. Convection PCR, and a suitable device for classical PCR, are described in Wheeler et al. (2004) “Convectively Driven Polymerase Chain Reaction Thermal Cycler,” 76:4011-4016. FIG. 13A shows an illustration of this device, and FIG. 13B shows how such a device can be designed for use in the method described herein.

### Kits

Also contemplated is a kit for carrying out the methods described herein. Such kits include one or more reagents useful for practicing any of these methods. A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the screening methods described herein. Instructions included in kits can be affixed to packaging material or can be included as a package insert. While the instructions are typically written or printed materials they are not limited to such. Any medium capable of storing such instructions and communicating them to an end user can be employed. Such media include, but are not limited to, electronic storage media (e.g., magnetic discs, tapes, cartridges, chips), optical media (e.g., CD ROM), and the like.

As used herein, the term “instructions” can include the address of an internet site that provides the instructions.

### EXAMPLES

### Example 1: Thermal Balancing of Primers, Probe, and Amplicon for Amplification in the Presence of Deoxyinosine Triphosphate

This Example describes an approach to selecting and designing primers, probes, and amplicons that are thermally balanced for use in amplifying a human genomic DNA target in the presence of dITP to reduce amplicon Tm.

For an illustrative thermal profile (fast toggle between 58° C. and 96° C.), we targeted primer Tms of ˜65° C.+/−2° C. and probe Tms of ˜70° C.+/−2° C. Tm calculations were estimated using G's instead of I's in the complementary strand when using inosine-based destabilized dNTPs, and we look for sequences where the primers would only contain 1-2 C's (if more C's are required, then the synthetic oligonucleotides [primers and/or probes] require more stabilization). Also, for the Tm estimates, we estimated the magnitude of the stabilization using the modified bases (i.e., T*, C*, etc., with the “*” indicating a stabilizing modification) as described below.

We used the following considerations in designing primers and a probe. Primer and probe Tms can be achieved by increasing the length of the oligos, looking for G-rich regions for the primers/probes (again, primers/probes have minimal Cs using dITP destabilization), or if there are numerous Cs, then we must increase the stabilizing modifications. Considering that longer a primer/probe is, the more likely the oligo will have more Cs, we utilized stabilizing chemistries (T*, C*, A*, etc) in the synthetic oligos to shorten the oligo length and intentionally tried to avoid C's in the primer/probe locations. We assumed a thermal stabilization of each T*/C* to be roughly 4 C and for A* ˜8 C for each modification. So a modified oligo was a best-guess estimate based on the parental natural sequence calculated and the adjustment for stabilization added (i.e., Tm with an additional 4 C for one T* modification or additional 8 C for two T*s modifications, etc.). Generally, primers had had </=2 Cs and probes had </=3 Cs when using the inosine destabilization.

Target amplicon Tms with inosine incorporation were around 64° C.-72° C. (measured experimentally via post-PCR melt with EvaGreen). The more stabilizing chemistry added to the primers, the higher the amplicon Tm since the stabilizing chemistry in primers gets retained in the amplicon. Generally, to achieve target amplicon Tms with inosine incorporated, we chose amplicons with G/C content ranging between 20%-40%, although higher G/C contents should be workable. In principle, the more G/C in the amplicon, the larger destabilization impact to the duplex when using dITP. We predict a G/C contents G/C content >40%, the amplicon Tₘ likely ends up near the target values of ˜70° C. (or perhaps slightly higher), but this depends on the length of the amplicon and how much stabilization chemistry is utilized in the primers. Additionally, the more G/Cs in the amplicon, the more difficult it can be to find appropriate locations for the primers and probes (i.e., with minimized C's in primer/probes for dITP). For our work, amplicon lengths were ˜65 to ˜85 bases. Amplicons can be longer, though longer amplicons would be expected to increase time-to-result (TTR), all other parameters being equal.

Under the conditions of our studies, we learned that the whole system of primers, probes and amplicon can be balanced thermally by targeting a primer Tms of ˜65° C. (estimated with G [not I] in complement), a probe Tm of ˜70° C. (also estimated with G [not I] in complement) and an amplicon Tm of about ˜70° C. (experimentally determined with I in complement). Another consideration is avoiding too much stabilization of the primers as this will also stabilize the amplicon. Probes can be aggressively stabilized as they are not incorporated into the amplicon. When selecting the amplicon sequence, one attempts to ensure that the primers do not face too much destabilization because if the primers are over-stabilized, this may over-stabilize the amplicon. Over stabilization of the probe is less of a concern and could likely be substantially stabilized but not so much that this inhibits amplification.

### Example 2: Use of dITP in Polymerase Chain Reaction (PCR)

In this study, primers, probes, and amplicons designed according to Example 1 were used for amplification of human genomic DNA (hgDNA), Chlamydia trachomatis (Chlamydia) DNA, and Neisseria gonorrhoeae (Neisseria) DNA, and SARS-COV-2 (e.g., RdRP) RNA targets in the presence of dITP.

Primers and probes were stored in TE buffer. Illustrative primers and probe sequences targeted hgDNA, Chlamydia, Neisseria, and RdRP targets and had the sequences shown in Table 1.

TABLE 1
Primers and probes

| Oligonucleotide | Nucleotide Sequence | SEQ ID NO: |
|---|---|---|
| hgDNA Forward primer | 5′ AAAAT*TTAGAAATATAATTGACAGGA 3′ | (SEQ ID NO: 1) |
| hgDNAReverse primer | 5′ GAAGT*AAATATGAATCTTATATGAC 3′ | (SEQ ID NO: 2) |
| hgDNA Probe (labeled | 5′ GGAAATTTGT*TATAATGAATGAAAC 3′ | (SEQ ID NO: 3) |
| with FAM and Q3) | | |
| Chlamydia forward primer | 5′ TTTGGT*GTGAGAGTTTAGG 3′ | (SEQ ID NO: 4) |
| Chlamydia reverse primer | 5′ TTTGCGAAAAGATGGAAT*T 3′ | (SEQ ID NO: 5) |
| Chlamydia probe (labeled | 5′ TA*A*GGAAA*GCA*GTT*CGGCA*G 3′ | (SEQ ID NO: 6) |
| with TxR and Q3) | | |
| Neisseria gonorrhoeae | 5′ GGTATTGAAGGTAAGGCTG 3′ | (SEQ ID NO: 7) |
| forward primer | | |
| Neisseria gonorrhoeae | 5′ TGGAGCAAAT*GGATGGAT*GA 3′ | (SEQ ID NO: 8) |
| reverse primer | | |
| Neisseria gonorrhoeae | 5′ GATGTCGAT*TATCGGT*ATTC 3′ | (SEQ ID NO: 9) |
| probe (labeled with cy5 | | |
| and Q) | | |
| RdRP forward primer | 5′ ATGATATCGTAAAAACAGATGGT*A 3′ | (SEQ ID NO: 10)) |
| RdRP reverse primer | 5′ GATGTTTAGTAAGTGGGT*AA 3′ | (SEQ ID NO: 11)) |
| RdRP probe (labeled with | 5′ TGATTGAACGGT*TCGTGTCTTT*AG 3′ | (SEQ ID NO: 12)) |
| Alexa532 and Q) | | |

Where T* refers to a T modified to increase stability, and A* refers to an A modified to increase stability.

### Single-Plex PCR

A single-plex PCR reaction was prepared containing Tris pH 9 at 20 mM, KCl at 40 mM, MgCl2 at 4 mM, Tween-20 at 0.5%, polymerase (like Roche AptaTaq) at 0.3 U/μl, 250 μM dCTP, 250 μM dATP, 250 μM dTTP, 250 μM dITP (or dGTP), forward primer at 250 nM, reverse primer at 250 nM, probe at 250 nM (or 1× EvaGreen for amplicon Tₘ analysis), and 10,000 copies/reaction of human genomic DNA. PCR was performed in 10 μl reactions on PCRmax Eco48 using a thermal profile of 95° C. for 8 seconds and 60° C. 30 seconds repeated for 45 cycles.

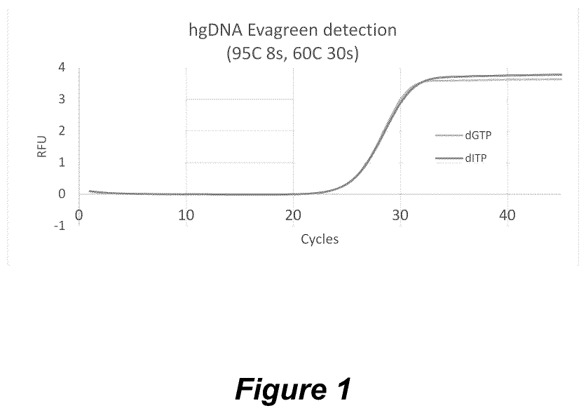

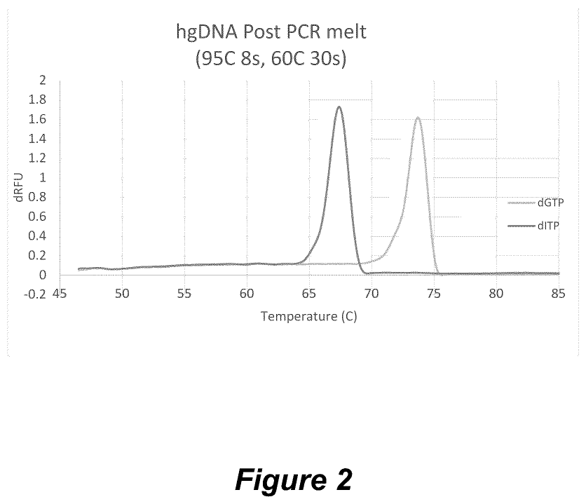

The PCR results using routine thermal cycling conditions show near-equal performance in terms of Ct and end point fluorescence when reaction uses dGTP or dITP (FIG. 1). The main difference is seen in the post-PCR melt, where the amplicon Tₘ observed for the dITP reaction is ˜67.5° C., whereas the amplicon Tₘ observed for the dGTP reaction is ˜73.9° C. (FIG. 2).

A single-plex PCR reaction for fast cycling was prepared containing Tris pH 9 at 20 mM, KCl at 40 mM, MgCl2 at 4 mM, Tween-20 at 0.5%, polymerase (like Roche AptaTaq) at 0.3 U/μl, 250 μM dCTP, 250 μM dATP, 250 μM dTTP, 250 μM dITP (or dGTP), forward primer at 250 nM, reverse primer at 250 nM, probe at 250 nM (or 1× EvaGreen for amplicon Tₘ analysis), and 10,000 copies/reaction of human genomic DNA. PCR was performed in 10 μl reactions on a PCRmax Eco48 using a thermal profile of 77° C. for 1 seconds and 58° C. 1 second repeated for 50-100 cycles.

The PCR data show amplification only in the reaction containing dITP. The lower denaturation temperature of 77° C. can only denature the amplicon with inosine incorporated (FIG. 3). The lower denaturation temperature, in combination with the reduction in hold times, results in an overall decrease in time per cycle. In this case, the cycle time was ˜15 s including time for heat to ramp up/down, hold time at each temperature, and optical read. In this instance, PCR efficiency was reduced since denature temperature and hold times were reduced, but the reduction in time per cycle results in an overall reduction in time to result.

### Multiplex PCR—Two Targets

A multiplex PCR reaction to detect hgDNA and Chlamydia DNA targets was prepared. For multiplex PCR with fast amplification cycles, a higher concentration of all primers and polymerase was used.

The multiplex PCR reaction contained Tris pH9 at 20 mM, KCl at 40 mM, MgCl2 at 4 mM, Tween-20 at 0.5%, polymerase (like Roche AptaTaq) at 2 U/μl), 250 μM dCTP, 250 μM dATP, 250 μM dTTP, 250 μM dITP or dGTP, forward primers for all targets at 800 nM, reverse primers for all targets at 800 nM, probes for all targets at 800 nM, and 10,000 copies/reaction of purified DNA for all targets. In one protocol, PCR is performed using the minimum setting at denaturation and extension temperatures. PCR is performed in 10 μl reactions on a PCRmax Eco48 using a thermal profile of 95° C. for 1 second and 60° C. for 1 second repeated from 50-100 cycles. We found that lower denaturation temperatures could be used to shorten time-to-result. Faster cycles can be achieved with instruments with faster thermal ramp rates, like the Cepheid GeneXpert® instrument. Faster cycles can also be achieved by removing the requirement for the instrument to reach the setpoint before starting the hold time. In the present example, thermal cycling was performed on a GeneXpert with 95° C. for about 10 seconds, about 58° C. for about 10 seconds, followed by cycles of about 96° C. for about 2 seconds and about 58° C. for about 5 seconds.

The data shown in FIG. 4 use Mastermix containing all primers and probes for both targets, dITP as a replacement for dGTP, hgDNA, and Chlamydia DNA. (Data not shown indicated no signal for a no-template control reaction and amplification of hgDNA and Chlamydia DNA in separate reactions where these were the only types of DNA present.) Therefore, this study demonstrated both selective and simultaneous amplification and detection of hgDNA and Chlamydia trichomonas.

### Multiplex PCR-Three Targets at Various Copy Levels

A multiplex PCR reaction was prepared to detect hgDNA, Chlamydia DNA, and Neisseria DNA targets.

The multiplex PCR reaction contained Tris pH9 at 20 mM, KCl at 40 mM, MgCl2 at 4 mM, Tween-20 at 0.5%, polymerase (like Roche AptaTaq) at 2 U/μ1, 250 μM dCTP, 250 μM dATP, 250 μM dTTP, 250 μM dITP or dGTP, forward primers for all targets at 800 nM, reverse primers for all targets at 800 nM, probes for all targets at 800 nM, and 10,000, 2000, 400, 80, or 16 copies/reaction of purified DNA for all targets. DNA concentration was used as given by the manufacturer, and serially diluted for various concentrations. (Assumed accurate quantitation and dilution). In one protocol, PCR is performed using the minimum setting at denaturation and extension temperatures. PCR is performed in 10 μl reactions on a PCRmax Eco48 using a thermal profile of 95° C. for 1 second and 60° C. for 1 second repeated from 50-100 cycles. We found that lower denaturation temperatures could also be used to further shorten time-to-result. Faster cycles can be achieved with instruments with faster thermal ramp rates, like the Cepheid GeneXpert® instrument. Faster cycles can also be achieved by removing the requirement for the instrument to reach the setpoint before starting the hold time. In the present example, thermal cycling was performed on a GeneXpert with 95° C. for about 10 seconds, about 58° C. for about 10 seconds, followed by cycles of about 96° C. for about 2 seconds and about 58° C. for about 5 seconds.

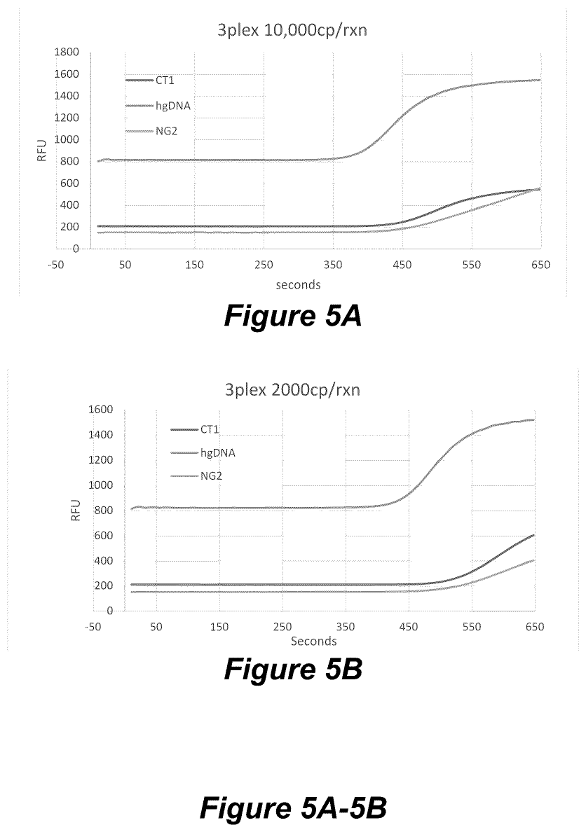

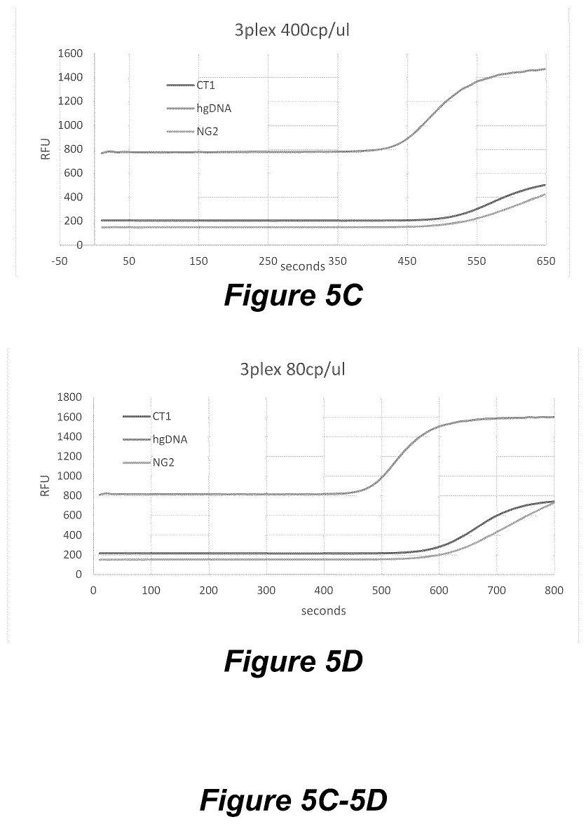

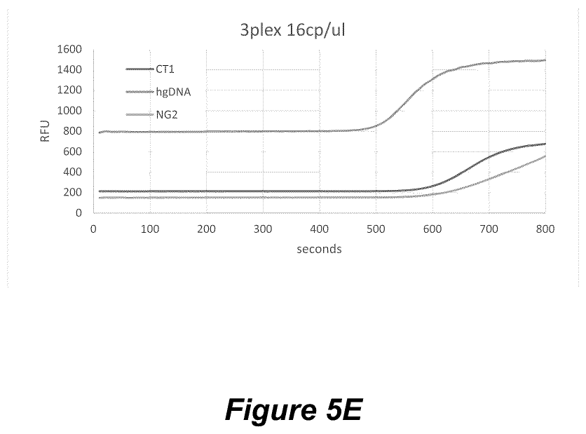

The results, shown in FIGS. 5A-5E, demonstrate the detection of all three targets down to a concentration of about 16 copies per microliter. (Data not shown indicated no signal for a no-template control reaction and amplification of hgDNA, Chlamydia DNA, and Neisseria DNA targets in separate reactions where these were the only types of DNA present.)

### Single-Plex RT-PCR

A singleplex RT-PCR reaction for RdRP was prepared. The reaction contained Tris pH9 at 20 mM, KCl at 40 mM, MgCl2 at 4 mM, Tween-20 at 0.5%, polymerase (like Roche AptaTaq) at 2 U/μl), reverse transcriptase (like Roche NxtScript 2G at 6.2 U/μl), 250 μM dCTP, 250 μM dATP, 250 μM dTTP, 250 μM dITP or dGTP, RdRP forward primer 800 nM, RdRP reverse primer at 800 nM, RdRP probe at 800 nM, and 625 copies/reaction of purified SARS-COV-2 RNA. The reverse transcription step was performed at 55° C. for 120 s. A 95 C 10 s hold, then 58° C. 10 s hold was performed, followed by PCR cycling. PCR was performed using the minimum setting at denaturation and extension temperatures. Thermal cycling was performed on GeneXpert with 95° C. for about 10 seconds, about 58° C. for about 10 seconds, followed by cycles of about 96° C. for about 2 seconds and about 58° C. for about 5 seconds.

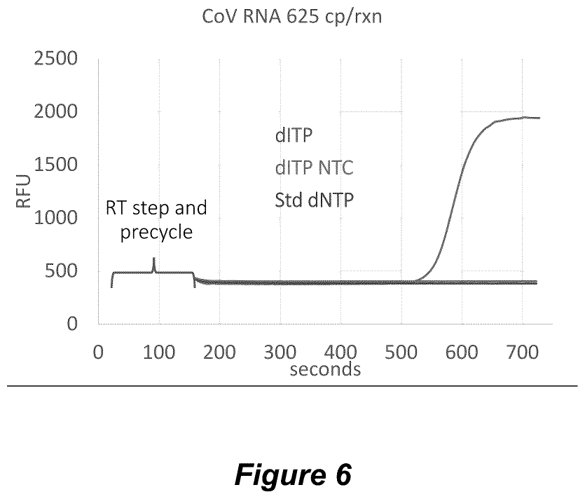

The data are shown in FIG. 6 for a reaction having mastermix containing primers and probes for RdRP, and dITP as a replacement for dGTP. The data demonstrates RT-PCR detection of SARS-COV-2 RNA. No amplification is observed when no target is present (NTC) or when standard dNTPs are used.

### Example 3: Effect of Inosine Incorporation on Oligonucleotide Tm

Melting temperatures for primer and probe sequences were measured against their complimentary sequences. The complimentary sequences were synthesized with canonical bases or replacement of G with inosine. Oligonucleotides and their complements were subjected to a thermal melt and slow cooling, followed by slow temperature increase to observe Tₘ. In the case of primers, the compliment was synthesized with a dye and quencher. In the case of probes, the compliment was left unlabeled.

A reaction was prepared containing Tris pH9 at 20 mM, KCl at 40 mM, MgCl2 at 4 mM, Tween-20 at 0.5%, primer/probe at 250 μM, and its compliment at 250 μM. Melt analysis was performed in 10 μl reactions on a PCRmax Eco48 by heating the reaction to 95° C. for 15 s, then cooling to 40° C. over 165 s, then melting by slowly raising the temperature to 95° C. The oligonucleotides tested are shown in the table below, along with the measured Tm's.

TABLE 2
Oligonucleotides for Tm analysis

| ID | Sequence | Tm | SEQ ID NO: |
|---|---|---|---|
| Neisseria gonorrhoeae | 5′ GGTATTGAAGGTAAGGCTG 3′ | — | (SEQ ID NO: 7) |
| forward primer | | | |
| Neisseria gonorrhoeae | 5′ CAGCCTTACCTTCAATACC 3′ | 62.6 | (SEQ ID NO: 13) |
| forward primer | | | |
| compliment with G | | | |
| (labeled with dye and Q | | | |
| Neisseria gonorrhoeae | 5′ CAICCTTACCTTCAATACC 3′ | 60.1 | (SEQ ID NO: 14) |
| forward primer | | | |
| compliment with I (labeled | | | |
| with dye and Q) | | | |
| Neisseria gonorrhoeae | 5′ TGGAGCAAAT*GGATGGAT*GA 3′ | — | (SEQ ID NO: 8) |
| reverse primer | | | |
| Neisseria gonorrhoeae | 5′ TCATCCATCCATTTGCTCCA 3′ | 73.1 | (SEQ ID NO: 15) |
| reverse primer | | | |
| compliment with G | | | |
| (labeled with dye and Q) | | | |
| Neisseria gonorrhoeae | 5′ TCATCCATCCATTTICTCCA 3′ | 70.4 | (SEQ ID NO: 16) |
| reverse primer | | | |
| compliment with I (labeled | | | |
| with dye and Q) | | | |
| Neisseria gonorrhoeae | 5′ GATGTCGAT*TATCGGT*ATTC 3′ | — | (SEQ ID NO: 9) |
| probe (labeled with dye | | | |
| and Q) | | | |
| Neisseria gonorrhoeae | 5′ GAATACCGATAATCGACATC 3′ | 67.1 | (SEQ ID NO: 17) |
| probe compliment with G | | | |
| Neisseria gonorrhoeae | 5′ IAATACCIATAATCIACATC 3′ | 58.1 | (SEQ ID NO: 18) |
| probe compliment with I | | | |

Where T* refers to a T modified to increase stability.

### Example 4: Use of dITP in Isothermal Amplification

An isothermal amplification reaction was prepared containing Tris pH9 at 20 mM, KCl at 40 mM, MgCl2 at 4 mM, Tween-20 at 0.5%, polymerase (like Roche AptaTaq) at 2 U/μl), 250 μM dCTP, 250 M μdATP, 250 μM dTTP, 250 μM dITP or dGTP, forward primers for all targets at 250 nM, reverse primers for all targets at 250 nM, 1× EvaGreen, and 10,000 copies/reaction of purified hgDNA. PCR was performed in 10 μl reactions on PCRmax Eco48 using a thermal profile of 95° C. for 8 s, 60° C. for 30 s, 95° C. for 8 s, then holding isothermally at 65° C. for 1 hr or longer. This isothermal hold was programmed by setting a 65° C. hold for 50 s and repeating this step several times.

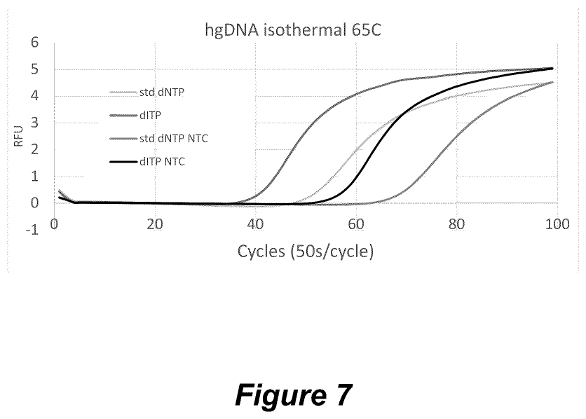

The results (FIG. 7) show signal generation at around 33 mins when using dITP, about 5 mins faster than the reaction using dGTP. The isothermal hold temperature of 65° C. was optimized around the amplicon Tm. NTCs appear several minutes delayed behind the +target reaction. Isothermal performance could be further increased by use of a different enzyme, for example BST which possesses strand displacement activity.

### Example 5: Use of 7-Deaza-dITP in Polymerase Chain Reaction

7-deaza-dITP can be used as a replacement for dGTP. The removal of the exocyclic amine gives a reduced Tm similar to dITP. Removal of the nitrogen at the 7 position can give an additional reduction in amplicon secondary structure. This can result in increased PCR efficiency.

A single-plex PCR reaction was prepared containing Tris pH 9 at 20 mM, KCl at 40 mM, MgCl2 at 4 mM, Tween-20 at 0.5%, polymerase (like Roche AptaTaq) at 0.3 U/μl, 250 μM dCTP, 250 μM dATP, 250 μM dTTP, 250 μM 7-deaza-dITP (or dGTP, or dITP), forward primer at 250 nM (SEQ ID NO:19), reverse primer at 250 nM (SEQ ID NO:2), probe at 250 nM (SEQ ID NO:3) (or 1× EvaGreen for amplicon Tm analysis), and 10,000 copies/reaction of human genomic DNA. In one protocol, PCR is performed in 10 μl reactions on a PCRmax Eco48 using a thermal profile of 95° C. for 8 seconds and 60° C. 30 seconds repeated for 40 cycles. In the present example, thermal cycling was performed on GeneXpert with 95° C. for about 10 seconds, about 58° C. for about 10 seconds, followed by cycles of about 96° C. for about 2 seconds and about 58° C. for about 5 seconds.

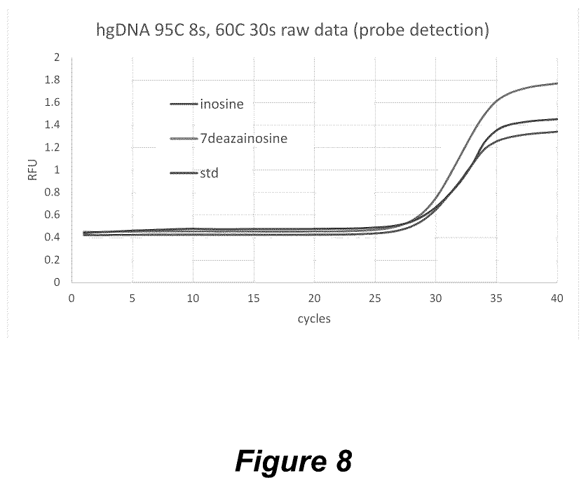

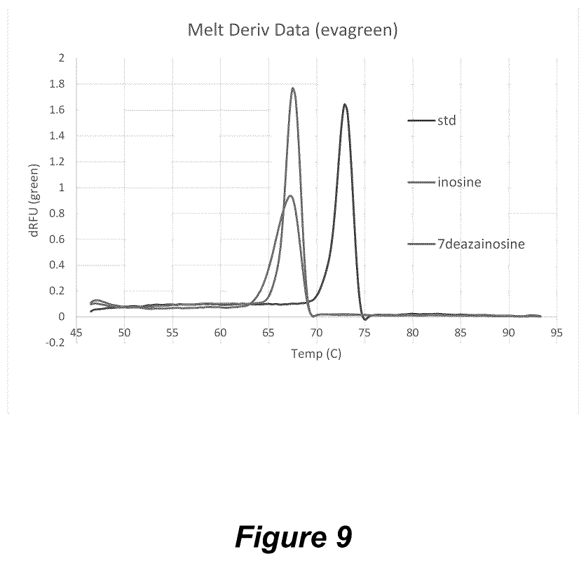

The results, shown in FIG. 8, indicate comparable Ct and endpoint fluorescence between dGTP and dITP, and slightly better results with 7-deaza dITP. Amplicon Tm analysis, shown in FIG. 9, demonstrates that the Tm reduction compared to dGTP is similar between both dITP and 7-deaza-dITP. The eduction in amplicon melt peak height could be attributed to EvaGreen dye quenching in the presence of 7-deaza-dITP.

Using the same reaction mix as above, PCR was performed using short wait times at denaturation and extension temperatures. PCR was performed in 10 μl reactions on a PCRmax Eco48 using a thermal profile of 95° C. for 1 second and 60° C. for 1 second repeated from 50-100 cycles. We found that lower denaturation temperatures could also be used to further shorten time-to-result. Faster cycles can be achieved with instruments with faster thermal ramp rates, like the Cepheid GeneXpert® instrument. Faster cycles can also be achieved with by removing the requirement for the instrument to reach the setpoint before starting the hold time.

The results shown in FIG. 10 indicate a slightly delayed detection time, but with a ˜30% increase in endpoint fluorescence. An increase in endpoint fluorescence could be attributed to the more favorable oligonucleotide kinetics when the amplicon has decreased secondary structure. The delay in detection time could be attributed to reduced affinity between the polymerase and 7-deaza-dITP. These effects are observed more readily when annealing times are low.

1. A kit comprising:
at least one destabilizing dNTP; and
a primer pair for amplifying a target nucleic acid in the presence of the at least one destabilizing dNTP, wherein each primer:
comprises no more than four, three, two, or one canonical bases that hybridize to the base corresponding to the destabilizing dNTP; and
optionally comprises a modification to increase the stability of a duplex formed upon hybridization, relative to an unmodified primer.

2. The kit of claim 1, wherein the modification is that each primer comprises at least one stabilizing base.

3. The kit of claim 1, wherein the modification is that each primer comprises one or more peptide nucleic acid, one or more locked nucleic acid, and one or more minor groove binder.

4. The kit of claim 1, wherein the at least one destabilizing dNTP is provided in a mixture of destabilizing dNTP with the canonical dNTP corresponding to the destabilizing dNTP in a ratio of greater than 1:1, optionally greater than about: 5:1, 10:1, 15:1, 20:1, 25:1, 30:1, or 35:1 destabilizing dNTP: canconical dNTP.

5. The kit of claim 1, wherein the kit does not comprise the canonical dNTP corresponding to the destabilizing dNTP.

6. The kit of claim 1, wherein each primer has a Tm of from about 63 degrees C. to about 67 degrees C., optionally about 65 degrees C., when hybridized to a complementary sequence of canonical bases.

7. The kit of claim 1, wherein the primer pair defines an amplicon that has a Tm of from about 60 degrees C. to about 75 degrees C., optionally about 70 degrees C., when measured after amplification and incorporation of the at least one destabilizing base.

8. The kit of claim 1, wherein the primer pair defines an amplicon that has a G-C content of less than 50%, optionally less than 40%.

9. The kit of claim 1, wherein the primer pair defines an amplicon that has a length of about 50 bases to about 100 bases, optionally about 65 bases to about 85 bases.

10. The kit of claim 1, wherein the kit comprises a probe.

11. The kit of claim 10, wherein the probe comprises no more than six, five, four, three, or 2 canonical bases that hybridize to the base corresponding to the destabilizing dNTP.

12. The kit of claim 10, wherein the probe comprises at least one stabilizing base.

13. The kit of claim 2, wherein each primer comprises at least 2, optionally 3 or 4 stabilizing bases, and/or each probe comprises at least 2, optionally 3, 4, or 5 stabilizing bases.

14. The kit of claim 2, where the stabilizing base is selected from 2-aminoadenine, C-5 propynyl-dC, C-5 propynyl-dU, 5-methyl-dC, and a locked nucleic acid base.

15. The kit of claim 10, wherein the probe has a Tm of from about 60 degrees C. to about 80 degrees C., optionally about 70 degrees C., when hybridized to a complementary sequence of canonical bases.

16-17. (canceled)

18. A method of selecting and/or designing a target nucleic acid from which an amplicon is produced upon nucleic acid amplification in the presence of a destabilizing dNTP, a primer pair capable of producing the amplicon, and an optional probe capable of detecting the amplicon, wherein the amplicon, primers, and probe are thermally balanced to promote amplification of an amplicon having a lower Tm than that of a double-stranded form of the target nucleic acid, prior to amplification, wherein the method comprises:
selecting the target nucleic acid sequence, primers, and optional probe so that destabilizing bases are incorporated into the amplicon outside of primer and/or probe target sequences in the amplicon;
if one or more primers will face one or more destabilizing bases in the amplicon, modifying the primer to increase the stability of a duplex formed upon hybridization, relative to an unmodified primer; and/or
if a probe that will face one or more destabilizing bases is included, modifying the probe to increase the stability of a duplex formed upon hybridization.

19. A method for amplifying a target nucleic acid using the components of the kit of claim 1, wherein the method comprises contacting the primers with sample nucleic acids under conditions suitable for amplification, such conditions comprising the presence of the at least one destabilizing dNTP, wherein the amplification produces an amplicon that has a lower Tm than that of a double-stranded form of the target nucleic acid, prior to amplification.

20. The method of claim 19, wherein the destabilizing dNTP is present in an amplification reaction mixture a ratio of greater than about 1:1, optionally about: 5:1, 10:1, 15:1, 20:1, 25:1, 30:1, or 35:1 destabilizing dNTP: canonical dNTP.

21. The method of claim 19, wherein the method does not employ any of the canonical dNTP corresponding to the destabilizing dNTP.

22. The method of claim 19, wherein the amplification is carried out in multiplex.

23. The method of claim 19, wherein the amplification is selected from standard polymerase chain reaction (PCR), isothermal amplification, thermal convection PCR, base-3 PCR, base-6 PCR, and krypton amplification.

24-25. (canceled)

26. The method of claim 19, wherein thermal cycling is carried out with a difference between annealing and denaturation temperatures of less than about 50 degrees C., optionally less than about 45 degrees C., about 40 degrees C., about 35 degrees C., or about 30 degrees C.

27-28. (canceled)

29. The kit of claim 1, wherein the destabilizing dNTP is selected from 2-deoxyinosine-5-triphosphate (dITP), 7-deaza-2-deoxinosine-5-triphosphate, and N4-methyl dCTP (methyl dCTP).

30. The kit of claim 1, wherein the kit comprises, or the method employs, a component selected from the group consisting of a PCR enhancer, a volume-excluding reagent, and a polymerase faster than Taq polymerase.

31. The kit of claim 1, wherein the target nucleic acid is RNA, and the kit comprises or the method employs a reverse transcriptase.


SEQUENCE LISTING (18 sequences)

SEQ ID NO: 1 - DNA, 26 bp, synthetic construct
  source 1..26: mol_type = other DNA
  misc_feature 5: note = Nucleotide modified to increase the stability
aaaatttagaaatataattgacagga

SEQ ID NO: 2 - DNA, 25 bp, synthetic construct
  source 1..25: mol_type = other DNA
  misc_feature 5: note = Nucleotide modified to increase the stability
gaagtaaatatgaatcttatatgac

SEQ ID NO: 3 - DNA, 25 bp, synthetic construct
  source 1..25: mol_type = other DNA
  misc_feature 10: note = Nucleotide modified to increase the stability
ggaaatttgttataatgaatgaaac

SEQ ID NO: 4 - DNA, 19 bp, synthetic construct
  source 1..19: mol_type = other DNA
  misc_feature 6: note = Nucleotide modified to increase the stability
tttggtgtgagagtttagg

SEQ ID NO: 5 - DNA, 19 bp, synthetic construct
  source 1..19: mol_type = other DNA
  misc_feature 18: note = Nucleotide modified to increase the stability
tttgcgaaaagatggaatt

SEQ ID NO: 6 - DNA, 20 bp, synthetic construct
  source 1..20: mol_type = other DNA
  misc_feature 2: note = Nucleotide modified to increase the stability
  misc_feature 3: note = Nucleotide modified to increase the stability
  misc_feature 8: note = Nucleotide modified to increase the stability
  misc_feature 11: note = Nucleotide modified to increase the stability
  misc_feature 14: note = Nucleotide modified to increase the stability
  misc_feature 19: note = Nucleotide modified to increase the stability
taaggaaagcagttcggcag

SEQ ID NO: 7 - DNA, 19 bp, synthetic construct
  source 1..19: mol_type = other DNA
ggtattgaaggtaaggctg

SEQ ID NO: 8 - DNA, 20 bp, synthetic construct
  source 1..20: mol_type = other DNA
  misc_feature 10: note = Nucleotide modified to increase the stability
  misc_feature 18: note = Nucleotide modified to increase the stability
tggagcaaatggatggatga

SEQ ID NO: 9 - DNA, 20 bp, synthetic construct
  source 1..20: mol_type = other DNA
  misc_feature 9: note = Nucleotide modified to increase the stability
  misc_feature 16: note = Nucleotide modified to increase the stability
gatgtcgattatcggtattc

SEQ ID NO: 10 - DNA, 24 bp, synthetic construct
  source 1..24: mol_type = other DNA
  misc_feature 23: note = Nucleotide modified to increase the stability
atgatatcgtaaaaacagatggta

SEQ ID NO: 11 - DNA, 20 bp, synthetic construct
  source 1..20: mol_type = other DNA
  misc_feature 18: note = Nucleotide modified to increase the stability
gatgtttagtaagtgggtaa

SEQ ID NO: 12 - DNA, 24 bp, synthetic construct
  source 1..24: mol_type = other DNA
  misc_feature 12: note = Nucleotide modified to increase the stability
  misc_feature 22: note = Nucleotide modified to increase the stability
tgattgaacggttcgtgtctttag

SEQ ID NO: 13 - DNA, 19 bp, synthetic construct
  source 1..19: mol_type = other DNA
cagccttaccttcaatacc

SEQ ID NO: 14 - DNA, 19 bp, synthetic construct
  source 1..19: mol_type = other DNA
  modified_base 3: mod_base = OTHER; note = Inosine
canccttaccttcaatacc

SEQ ID NO: 15 - DNA, 20 bp, synthetic construct
  source 1..20: mol_type = other DNA
tcatccatccatttgctcca

SEQ ID NO: 16 - DNA, 20 bp, synthetic construct
  source 1..20: mol_type = other DNA
  modified_base 15: mod_base = OTHER; note = Inosine
tcatccatccatttnctcca

SEQ ID NO: 17 - DNA, 20 bp, synthetic construct
  source 1..20: mol_type = other DNA
gaataccgataatcgacatc

SEQ ID NO: 18 - DNA, 20 bp, synthetic construct
  source 1..20: mol_type = other DNA
  modified_base 1: mod_base = OTHER; note = Inosine
  modified_base 8: mod_base = OTHER; note = Inosine
  modified_base 15: mod_base = OTHER; note = Inosine
naataccnataatcnacatc